(19) United States
(12) Patent Application Publication
(10) Pub. No.: US 2004/0175744 A1
(43) Pub. Date: Sep. 9, 2004

(54) Polynucleotide sequences and corresponding encoded polypeptides of particular secreted and membrane-bound proteins overexpressed in certain cancers

(76) Inventors: Qianjin Hu, Castro Valley, CA (US); Alan Peng, Palo Alto, CA (US); Bin Liu, Castro Valley, CA (US); James Robert Love, Millbrae, CA (US); Xiaofeng Hao, Chengdu (CN); Min Ren, Chengdu (CN); Zhumei Sheng, Chengdu (CN)

Correspondence Address: Dr Jamie Love, Vice President of Business Development, 14826 Wicks Blvd, San Leandro, CA, 94577, US

(21) Appl. No.: 10/775,920

(22) Filed: Feb. 10, 2004

Related U.S. Application Data
(60) Provisional application No. 60/447,900, filed on Feb. 13, 2003.

(51) Int. Cl. C12Q 1/68; G01N 33/53; G01N 33/574

(52) U.S. Cl. 435/6; 435/7.23

(57) ABSTRACT

The present invention relates to the identification of the over expression of certain genes encoding certain secreted and membrane-bound proteins in breast, lung, colon and kidney cancers. Knowledge that these genes are over expressed in the cancers is useful for the detection, diagnosis, prevention and treatment of the specific cancers.

### CROSS-REFERENCE TO RELEATED APPLICATION

This application claims priority benefit to U.S. provisional application Ser. No. 60/447,900, filed Feb. 13, 2003, for “POLYNUCLEOTIDE SEQUENCES AND CORRESPONDING ENCODED POLYPEPTIDES OF PARTICULAR SECRETED AND MEMBRANE-BOUND PROTEINS OVEREXPRESSED IN CERTAIN CANCERS”, which is incorporated herein in its entirety. The corresponding PCT application, for which an application number is not yet granted, has been filed and is also incorporated herein by reference.

### FEDERALLY SPONSORED RESEARCH

Not Applicable.

### SEQUENCE LISTING/FILE

All sequences referenced in this patent are on CD with file “14dec03.ST25.txt” (generated by PatentIn3.2) attached to this application and incorporated by reference.

### TECHNICAL FIELD OF THE INVENTION

This invention relates to certain polynucleotides encoding certain secreted and membrane-bound proteins (and the polypeptides they encode) which have been newly identified in connection with particular cancers; and uses of such polynucleotides and polypeptides in the detection, diagnosis, prevention and/or treatment of cancer.

### BACKGROUND OF THE INVENTION

Proteins targeted to the ER by a signal sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins are stored in secretory vesicles (or secretory granules) until exocytosis is triggered. Similarly, proteins residing on the cell membrane can also be secreted into the extracellular space by proteolytic cleavage of a “linker” holding the protein to the membrane.

Proteins lodged in the membrane of a cell (membrane-bound proteins) can facilitate signaling in the cell by being bound by a ligand and signaling through the interior portion of the protein activity to occur in the cell, for example via signal transduction mechanisms. Examples of such proteins are member for the tumor necrosis factor receptor family and the G-protein receptor family.

Both secreted and membrane-bound proteins are active in cell function and cell-cell communications and ultimately facilitate the well-being of a cell or its disorder. Recent progress in gene discovery has furthered interest in gene function, and two important functional groups are secreted proteins and membrane-bound proteins.

Thus, in light of the pervasive role of secreted and membrane-bound proteins in human physiology, a need exists for identifying and characterizing these proteins in the context of disorders such as cancer so that knowledge of the aberrant expression of such a protein in a particular cancer can be used to both diagnose and treat the cancer. The described invention identifies genes whose differential-regulation (increase in transcription and translation) is identified in tumors versus healthy tissue. This information (invention) can be used to arrive at a better diagnosis, prognosis or treatment regime. In addition, this information (invention) can be used to develop treatments or prevention such as (but not limited to) antisense technology, interfering RNA (iRNA) methods, antibodies or other ligands (with or without added effector functions), or as a foundation upon which to develop other therapeutic methods such as gene therapy methods.

### SUMMARY OF THE INVENTION

The present invention relates to diagnostic, prevention and therapeutic methods for detecting and treating certain cancers.

The present invention provides a method of breast cancer diagnosis comprising detecting over expression of a gene of a protein found in breast tissue selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDR/TAP) and Type I transmembrane receptor (seizure-related protein).

The gene can comprise a sequence selected from the group consisting of SEQ ID Nos 1-154.

In the method, detecting over expression can comprise a method selected from the group consisting of detecting a level of mRNA expression, detecting a level of protein expression, and probing for a polypeptide encoded by the gene.

The over expressed protein can comprise an amino acid sequence encoded by a gene selected from the group of genes having nucleic acid sequences of SEQ ID Nos 1-154.

The invention further provides a method of breast cancer treatment comprising blocking over expression of a gene encoding a secreted or membrane-bound protein found in breast tissue selected from the group consisting of SEQ ID Nos 1-154.

The gene can comprise a sequence selected from the group consisting of SEQ ID Nos 1-154.

The method of treatment can comprise a method selected from the group consisting of blocking expression of an mRNA encoded by the gene, blocking translation of the mRNA encoded by the gene, blocking expression of a protein encoded by the gene, blocking the function of the expressed protein, promoting the degradation of the expressed protein and using the protein as a tag to deliver treatment.

The method of treatment can comprise blocking an mRNA, polypeptide or protein comprising a nucleotide or amino acid sequence encoded by a gene selected from the group of genes having nucleic acid sequences of SEQ ID Nos 1-154.

The over expressed protein can be secreted.

The over expressed protein can be membrane-bound.

The invention further provides a method of lung cancer diagnosis comprising detecting over expression of a gene of a protein found in lung tissue selected from the group consisting of hypothetical protein, amino acid transporter 2, carboxypeptidase M, and putative G-protein coupled receptor.

The gene can comprise a sequence selected from the group consisting of SEQ ID Nos 155-184.

Detecting over expression of the gene comprises a method selected from the group consisting of detecting a level of mRNA expression, detecting a level of protein expression, and probing for a polypeptide encoded by the gene.

The over expressed protein can comprise an amino acid sequence encoded by a gene selected from the group of genes having nucleic acid sequences of SEQ ID Nos 155-184.

The invention also provides a method of lung cancer treatment comprising blocking over expression of a gene encoding a secreted or membrane-bound protein found in lung tissue selected from the group consisting of SEQ ID Nos 155-184.

The gene can comprise a sequence selected from the group consisting of SEQ ID Nos 155-184.

The method of treatment can comprise a method selected from the group consisting of blocking expression of an mRNA encoded by the gene, blocking translation of the mRNA encoded by the gene, and blocking expression of a protein encoded by the gene, blocking the function of the expressed protein, promoting the degradation of the expressed protein and using the protein as a tag to deliver treatment.

The method of treatment can comprise blocking an mRNA, polypeptide or protein comprising a nucleotide or amino acid sequence encoded by a gene selected from the group of genes having nucleic acid sequences of SEQ ID Nos 155-184.

The over expressed protein can be secreted.

The over expressed protein can be membrane-bound.

The invention further provides a method of colon cancer diagnosis comprising detecting over expression of a gene of a protein found in colon tissue selected from the group consisting of hypothetical protein (colon), cadherin, formyl peptide receptor 1, AE-binding protein 1, solute carrier family 21 (organic anion transporter) member 12, secreted phosphoprotein 1 (osteopontin, bone sialoprotein I), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

The gene can comprise a sequence selected from the group consisting of SEQ ID Nos 185-246.

Detecting over expression can comprise a method selected from the group consisting of detecting a level of mRNA expression, detecting a level of protein expression, and probing for a polypeptide encoded by the gene.

The over expressed protein can comprise an amino acid sequence encoded by a gene selected from the group of genes having nucleic acid sequences of SEQ ID Nos 185-246.

The invention also provides a method of colon cancer treatment comprising blocking over expression of a gene encoding a secreted or membrane-bound protein found in colon tissue selected from the group consisting of SEQ ID Nos 185-246.

The gene can comprise a sequence selected from the group consisting of SEQ ID Nos 185-246.

The method of treatment can comprise a method selected from the group consisting of blocking expression of an mRNA encoded by the gene, blocking translation of the mRNA encoded by the gene, and blocking expression of a protein encoded by the gene, blocking the function of the expressed protein, promoting the degradation of the expressed protein and using the protein as a tag to deliver treatment.

The method of treatment can comprise blocking an mRNA, polypeptide or protein comprising a nucleotide or amino acid sequence encoded by a gene selected from the group of genes having nucleic acid sequences of SEQ ID Nos 185-246.

The over expressed protein can be secreted.

The over expressed protein can be membrane-bound.

The invention further provides a method of kidney cancer diagnosis comprising detecting over expression of a gene of a protein found in kidney tissue selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150) alpha polypeptide), sema domain seven thrombospondin repeats (type 1 and type 1-like) transmembrane domain (TM) I and short cytoplasmic domain (semaphoring) 5B, Guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

The gene can comprise a sequence selected from the group consisting of SEQ ID Nos 247-385.

Detecting over expression can comprise a method selected from the group consisting of detecting a level OF mRNA expression, detecting a level of protein expression, and probing for a polypeptide encoded by the gene.

The over expressed protein can comprise an amino acid sequence encoded by a gene selected from the group of genes having nucleic acid sequences of SEQ ID Nos 247-385.

The invention also provides a method of colon cancer treatment comprising blocking over expression of a gene encoding a secreted or membrane-bound protein found in colon tissue selected from the group consisting of SEQ ID Nos 247-385.

The gene can comprise a sequence selected from the group consisting of SEQ ID Nos 247-385.

The method of treatment can comprise a method selected from the group consisting of blocking expression of AN mRNA encoded by the gene, blocking translation of THE mRNA encoded by the gene, and blocking expression of a protein encoded by the gene, blocking the function of the expressed protein, promoting the degradation of the expressed protein and using the protein as a tag to deliver treatment.

The method of treatment can comprise blocking AN mRNA, polypeptide or protein comprising a nucleotide or amino acid sequence encoded by a gene selected from the group of genes having nucleic acid sequences of SEQ ID Nos 247-385.

The over expressed protein can be secreted.

The over expressed protein can be membrane-bound.

The invention provides an antibody or binding portion of an antibody that specifically binds a protein found in breast tissue, wherein the protein is selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDRITAP) and Type I transmembrane receptor (seizure-related protein).

The invention further provides a method of breast cancer diagnosis or prognosis comprising contacting an antibody or binding portion of an antibody specific to an over expressed protein in breast cancer cells, with the over expressed protein for which the antibody or binding portion thereof is specific.

The invention also provides a method of breast cancer treatment comprising blocking an over expressed protein selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDRITAP) and Type I transmembrane receptor (seizure-related protein), with an antibody or binding portion thereof specific for said protein.

The invention likewise provides an antibody or binding portion of an antibody that specifically binds a protein found in lung tissue, wherein the protein is selected from the group consisting of over expressed in lung of hypothetical protein, amino acid transporter 2, carboxypeptidase M, and putative G-protein coupled receptor.

The invention also provides a method of lung cancer diagnosis or prognosis comprising contacting an antibody or binding portion of an antibody specific to an over expressed protein in lung cancer cells, with the over expressed protein for which the antibody or binding portion thereof is specific.

The invention provides a method of lung cancer treatment comprising blocking an over expressed protein selected from the group consisting of over expressed in lung of hypothetical protein, amino acid transporter 2, carboxypeptidase M, and putative G-protein coupled receptor, with an antibody or binding portion thereof specific for said protein.

The invention provides an antibody or binding portion of an antibody that specifically binds to a protein found in colon tissue, wherein the protein is selected from the group consisting of hypothetical protein (colon), cadherin, formyl peptide receptor 1, AE-binding protein 1, solute carrier family 21 (organic anion transporter) member 12, cadherin 3 type 1 P-cadherin (placental), secreted phosphoprotein 1 (osteopontin, bone sialoprotein I), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

The invention further provides a method of colon cancer diagnosis or prognosis comprising contacting an antibody or binding portion of an antibody specific to an over expressed protein in colon cancer cells, with the over expressed protein for which the antibody or binding portion thereof is specific

The invention also provides a method of colon cancer treatment comprising blocking an overexpressed protein selected from the group consisting of hypothetical protein (colon), cadherin, formyl peptide receptor 1, AE-binding protein 1, solute carrier family 21 (organic anion transporter) member 12, secreted phosphoprotein 1 (osteopontin, bone sialoprotein I), Type I transmembrane protein Fn14, hypoxia-inducible protein 2, alpha-2-glycoprotein 1 zinc, chitinase 3-like 1 (cartilage glycoprotein-39) and triggering receptor expressed on myeloid cells 2.

The invention provides an antibody or binding portion. of an antibody that specifically binds to a protein found in kidney tissue, wherein the protein is selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150), alpha polypeptide), sema domain seven thrombospondin repeats (type 1 and type 1-like) transmembrane domain (TM) and short cytoplasmic domain (semaphoring) 5B, Guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

The invention provides a method of kidney cancer diagnosis or prognosis comprising contacting an antibody or binding portion of an antibody specific to an over expressed protein in kidney cancer cells, with the over expressed protein for which the antibody or binding portion thereof is specific.

The invention provides a method of kidney cancer treatment comprising blocking an over expressed protein selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150), alpha polypeptide), sema domain seven thrombospondin repeats (type 1 and type 1-like) transmembranelain (TM) and and short cytoplasmic domain (semaphoring) 5B, Guanylate binding protein 1 interferon-inducible 67 kD, leukocyte immunoglobulin-like receptor subfamily B, discoidin domain receptor family member 2, caveolin 1 caveolaE protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

These and other objects and features of the invention will become more fully apparent when the following detailed description of the invention is read in conjunction with the accompanying drawings.

### BRIEF DESCRIPTION OF THE FIGS.

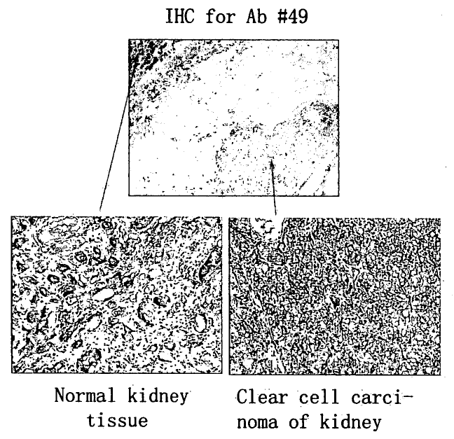

FIG. 1: Immunohistochemical data, produced using antibodies to Clone-49 (coding for Unigene#Hs.323477 and described as “solute carrier family 16 (monocarboxylic acid transporters), member 3; monocarboxylate transporter 3 [Homo sapiens]”), indicating evidence that the protein (“solute carrier family 16 (monocarboxylic acid transporters), member 3; monocarboxylate transporter 3 [Homo sapiens]”) is highly expressed on the membrane of the tumor (best seen at higher magnification in lower right image) but not normal tissues (best seen at higher magnification in lower left image).

### DETAILED DESCRIPTION OF PREFERRED EMBODIMENT

The present invention provides novel methods and compositions involving polypeptides and polynucleotides specifically differentially expressed in tissues of cancer patients as summarized in Table 1. The invention can be used in various aspects of genome analysis that finds utility in both basic biological research and medical diagnosis and therapeutics.

TABLE 1
Polynucleotides and Polypeptides of Secreted and Membrane-Bound Proteins
Previously Unidentified as Over Expressed in Breast, Lung, Colon and Kidney Cancers

| Mergen | Over expressed | | | |
|---|---|---|---|---|
| Clone | in Tumor | Sequence | UniGene ID# (or | |
| Identifier¹ | tissues | ID# | GeneBank#) | Description of gene |
| Clone-01 | breast | #1-8 | Hs.134514 | ATP-binding, transporter |
| Clone-02 | breast | #9-13 | Hs.194691 | GPCR (G-protein coupled receptor) |
| Clone-03 | breast | #14 | Hs.192720 | GPCR (G-protein coupled receptor) 14 |
| Clone-04 | breast | #15-16 | Hs.348504 | hypothetical protein (breast) |
| Clone-06 | breast | #17-20 | Hs.135187 | unc-93 |
| Clone-07 | breast | #21-23 | Hs.118110 | Bone marrow stromal cell antigen 2 |
| Clone-08 | breast | #24-27 | Hs.252387 | Cadherin, EGF LAG seven-pass G-type receptor 1, |
| | | | | flamingo (Drosophila) homolog (LAG = lymphocyte |
| | | | | activation protein) |
| Clone-09 | breast | #28-32 | Hs.11 | Carcinoembryonic antigen-related cell adhesion |
| | | | | molecule 3 |
| Clone-10 | breast | #33-37 | Hs.73848 | Carcinoembryonic antigen-related cell adhesion |
| | | | | molecule 6 (non-specific cross reacting antigen) |
| Clone-11 | breast | #38-42 | Hs.41 | Carcinoembryonic antigen-related cell adhesion |
| | | | | molecule 8 |
| Clone-12 | breast | #43-45 | Hs.278562 | Claudin 7 |
| Clone-15 | breast | #46-52 | Hs.106671 | Cleft lip and palate associated transmembrane protein 1 |
| Clone-16 | breast | #53-62 | Hs.1420 | Fibroblast growth factor receptor 3 (achondroplasia, |
| | | | | thanatophoric dwarfism) |
| Clone-17 | breast | #63-67 | Hs.202833 | Heme oxygenase (decycling) 1 |
| Clone-18 | breast | #68-76 | Hs.76095 | Immediate early response 3 |
| Clone-19 | breast | #77-94 | Hs.89603 | Mucin 1, transmembrane |
| Clone-20 | breast | #95-100 | Hs.334514 | NG22 protein |
| Clone-21 | breast | #101-108 | Hs.18079 | Phosphatidylinositol glycan, class Q |
| Clone-22 | breast | #109-119 | Hs.108408 | Similar to CGI-78 protein, clone MGC: 4880 |
| | | | | IMAGE:3457593, mRNA, complete cds |
| Clone-23 | breast | #120-125 | Hs.323878 | Solute carrier family 1 (glutamate/neutral amino acid |
| | | | | transporter), member 4 |
| Clone-24 | breast | #126-129 | Hs.187958 | Solute carrier family 6 (neurotransmitter transporter, |
| | | | | creatine), member 8 |
| Clone-25 | breast | #130-136 | Hs.38972 | Tetraspan 1 |
| Clone-26 | breast | #137-146 | Hs.352018 | Transporter 1, ATP-binding cassette, sub-family B |
| | | | | (MDR/TAP) |
| Clone-27 | breast | #147-154 | Hs.6314 | Type I transmembrane receptor (seizure-related protein) |
| Clone-28 | lung | #155-159 | Hs.283558 | Hypothetical protein |
| Clone-29 | lung | #160-170 | Hs.298275 | Amino acid transporter 2 |
| Clone-30 | lung | #171-175 | Hs.334873 | Carboxypeptidase M |
| Clone-31 | lung | #176-184 | Hs.16085 | Putative G-protein coupled receptor |
| Clone-13 | colon | #185 | (XM_085932) | NEW GENE |
| Clone-14 | colon | #186-188 | Hs.2877 | Cadherin |
| Clone-32 | colon | #189-193 | Hs.753 | Formyl peptide receptor 1 |
| Clone-33 | colon | #194-198 | Hs.118397 | AE-binding protein 1 |
| Clone-34 | colon | #199-206 | Hs.235782 | Solute carrier family 21 (organic anion transporter), |
| | | | | member 12 |
| Clone-35 | colon | #207-219 | Hs.313 | Secreted phosphoprotein 1 (osteopontin, bone |
| | | | | sialoprotein I, |
| Clone-36 | colon | #220-225 | Hs.355899 | Type I transmembrane protein Fn14 |
| Clone-37 | colon | #226-229 | Hs.61762 | Hypoxia-inducible protein 2 |
| Clone-38 | colon | #230-236 | Hs.71 | Alpha-2-glycoprotein 1, zinc |
| Clone-39 | colon | #237-241 | Hs.75184 | Chitinase 3-like 1 (cartilage glycoprotein-39) |
| Clone-40 | colon | #242-246 | Hs.44234 | Triggering receptor expressed on myeloid cells 2 |
| Clone-41 | kidney | #247-249 | Hs.180616 | Scavenger receptor class B |
| Clone-42 | kidney | #250-255 | Hs.258 | Adenosine receptor A3 |
| Clone-43 | kidney | #256-261 | Hs.3107 | CD97 antigen |
| Clone-44 | kidney | #262 | (M20903) | APO E-C2 gene |
| Clone-45 | kidney | #263-269 | Hs.10649 | Basement membrane-induced gene |
| Clone-46 | kidney | #270-273 | Hs.149609 | Integrin, alpha 5 (fibronectin receptor, alpha |
| | | | | polypeptide) |
| Clone-47 | kidney | #274-277 | Hs.237856 | Peptide transporter 3 |
| Clone-48 | kidney | #278-284 | Hs.25485 | Hypothetical protein FLJ22341 |
| Clone-49 | kidney | #285-286 | Hs.323477 | Solute carrier family 16 (monocarboxylic acid |
| | | | | transporters) |
| Clone-50 | kidney | #287-289 | Hs.327 | Interleukin 10 receptor, alpha |
| Clone-51 | kidney | #290-296 | Hs.333418 | FXYD domain-containing ion transport regulator 5 |
| Clone-52 | kidney | #297-301 | Hs.37682 | Retinoic acid receptor responder (tazarotene induced) 2 |
| Clone-53 | kidney | #302-306 | Hs.51077 | Integrin, alpha X (antigen CD11C (p150), alpha |
| | | | | polypeptide) |
| Clone-54 | kidney | #307-310 | Hs.61384 | Sema domain, seven thrombospondin repeats (type 1 |
| | | | | and type 1-like), transmembrane domain (TM) and short |
| | | | | cytoplasmic domain, (semaphorin) 5B |
| Clone-55 | kidney | #311-313 | Hs.62661 | Guanylate binding protein 1, interferon-inducible, 67 kD |
| Clone-56 | kidney | #314-319 | Hs.67846 | Leukocyte immunoglobulin-like receptor, subfamily B |
| Clone-57 | kidney | #320-322 | Hs.71891 | Discoidin domain receptor family, member 2 |
| Clone-58 | kidney | #323-330 | Hs.74034 | Caveolin 1, caveolae protein, 22 kD |
| Clone-59 | kidney | #331-335 | Hs.74276 | Chloride intracellular channel 1 |
| Clone-60 | kidney | #336-346 | Hs.75613 | CD36 antigen (collagen type I receptor, thrombospondin |
| | | | | recep) |
| Clone-61 | kidney | #347-354 | Hs.75703 | Small inducible cytokine A4 (homologous to mous Mip- |
| | | | | 1b) |
| Clone-62 | kidney | #355-358 | Hs.79356 | Lysosomal-associated multispanning membrane |
| | | | | protein-5 |
| Clone-63 | kidney | #359-365 | Hs.83968 | Integrin, beta 2 (antigen CD18 (p95), lymphocyte |
| | | | | funtion-assoc |
| Clone-64 | kidney | #366-375 | Hs.89414 | Chemokine (C-X-C motif), receptor 4 (fusin) |
| Clone-65 | kidney | #376-380 | Hs.89476 | CD2 antigen (p50), sheep red blood cell receptor |
| Clone-66 | kidney | #381-385 | Hs.9999 | Epithelial membrane protein 3 |

¹The 65 “clones” are numbered “Clone-01” to “Clone-66” but there is no clone “Clone-05“. Clone-13 and Clone-14 are repositioned in the table to better illustrate their tissue (tumor) association.

The present invention is based, in part, on the discovery of differential levels of expression of these listed polynucleotides in tissue samples of cancerous tissue, as compared to normal tissue samples. The present invention provides polynucleotides, as well as their corresponding gene products, that are present at elevated levels in a variety of tumors. These polynucleotides (or fragments thereof) and polypeptides (or antigenic fragments thereof), and antibodies that bind such polypeptides, are useful in a variety of diagnostic, prophylactic and therapeutic methods.

In other embodiments, a polynucleotide of the present invention comprises a detectable label, and/or is attached to a solid support. In other embodiments, a polynucleotide of the present invention is single stranded and in yet other embodiments, is double stranded. The present invention also encompasses host cells comprising an isolated polynucleotide of the present invention.

The present invention also provides kits, electronic libraries and arrays comprising polynucleotides of the present invention for use in diagnosing the presence of disease tissue in a test sample.

The present invention also provides methods of screening for disease activity, comprising: contacting a tissue sample derived from a cancer-associated cell with a drug candidate; monitoring the expression of a polynucleotide having the sequence shown in the file “14dec03.ST25.txt” (generated by PatentIn3.2) attached to this application and incorporated by reference or a fragment thereof, in the tissue sample; and determining the efficacy of the drug candidate.

Unless otherwise indicated, all terms used herein have the meanings given below, and are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F M et al. (1993) Current Protocols in Molecular Biology, John Wiley & Sons, New York, N.Y., for definitions and terms of the art. It is to be understood that this invention is not limited to the particular methodology, protocols, and reagents described, as these may vary.

All publications cited herein are expressly incorporated herein by reference for the purpose of describing and disclosing compositions and methodologies that might be used in connection with the invention.

1. Definitions, Methods and Materials

The following definitions are provided to facilitate understanding of certain terms used throughout this specification.

In the present invention, “isolated” refers to material removed from its original environment (e.g. the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” protein refers to those proteins capable of being directed to the ER, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

A “membrane-bound protein” refers to proteins such as described in U.S. Pat. Nos. 6,043,052 and 6,013,476, both of which describe exemplary membrane-bound proteins of two different families.

As used herein, a “polynucleotide” refers to a molecule having a nucleic acid sequence contained in the file “14dec03.ST25.txt” (generated by PatentIn3.2) attached to this application and incorporated by reference. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

In the present invention, the full-length sequence identified in the file “14dec03.ST25.txt” (generated by PatentIn3.2) attached to this application and incorporated by reference, was often generated by overlapping sequences contained in multiple clones (contig analysis).

A “polynucleotide” of the present invention also includes those polynucleotides capable of hybridizing, under stringent hybridization conditions, to sequences contained in the file “14dec03.ST25.txt” (generated by PatentIn3.2) attached to this application and incorporated by reference, the complement thereof. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH₂ PO₄; 0.02M EDTA, pH 7.4), 0.5% SDS, 30% formamide, 100 μg/ml salmon sperm blocking DNA; followed by washes at 50° C. with I×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+sequences (such as any 3′ terminal polyA+tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g. practically any double-stranded cDNA clone).

The polynucleotides of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotides, oligonucleotides primers or cDNA placed on a chip or other surfaces used to amplify, clone or identify target polynucleotides. Since the position of each particular group of oligonucleotides in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, plastics, polymers, membranes and silane or silicate supports such as glass slides.

As used herein, a “biological sample” refers to a sample of tissue or fluid isolated from an individual, including but not limited to, for example, blood, plasma, serum, spinal fluid, lymph fluid, the external sections of the skin, respiratory, intestinal, and genitourinary tracts, tears, saliva, milk, cells (including but not limited to blood cells), tumors, organs, and also samples of in vitro cell culture constituents.

The term “biological sources” as used herein refers to the sources from which the target polynucleotides are derived from. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

The polypeptides of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPl anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“DNA sequences” refers to a polynucleotide sequence identified by an integer specified in the file “14dec03.ST25.txt” (generated by PatentIn3.2) attached to this application and incorporated by reference.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

The over expressed genes detected from breast tissue include ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDR/TAP) and Type I transmembrane receptor (seizure-related protein).

The over expressed genes detected from lung tissue include hypothetical protein, amino acid transporter 2, carboxypeptidase M, and putative G-protein coupled receptor.

The over expressed genes detected from colon tissue include hypothetical protein (colon), cadherin, formyl peptide receptor 1, AE-binding protein 1, solute carrier family 21 (organic anion transporter) member 12, secreted phosphoprotein 1 (osteopontin, bone sialoprotein 1), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

The over expressed genes detected from kidney tissue include scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder.(tazarotene induced.) 2, integrin alpha X (antigen CD11C (p150), alpha polypeptide), sema domain seven thrombospondin repeats (type 1 and type 1-like) transmembrane domain (TM) and short cytoplasmic domain (semaphorin) 5B, guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

Table 1 identifies these genes by Sequence ID number and accession numbers and can be used as a reference to the file “14dec03.ST25.txt” (generated by PatentIn3.2) attached to this application and incorporated by reference.

Supplemental data supplied in Example 6 show the results supporting this patent by identifying the differential expression associated with diseased versus cancerous tissue.

Therefore, polynucleotides and polypeptides of the invention are useful as reagents for differential identification of the tissue(s) or cell type(s) present in a biological sample and for diagnosis of diseases and conditions which include, but are not limited to, breast cancer for some proteins, lung cancer for other proteins, colon cancer for other proteins, and kidney cancer for still other proteins. Some of these proteins fall into the category of secreted proteins and others are membrane-bound proteins. The proteins are referenced by the UniGene ID# (or GeneBank# when a UniGene ID# is not available) of their genes and the descriptive names of the proteins expressed by these genes. The polynucleotides are displayed sequentially in the Sequence listing in the file “14dec03.ST25.txt” (generated by PatentIn3.2) attached to this application and incorporated by reference.

Similarly, polypeptides and antibodies directed to these polypeptides are useful in providing immunological probes for differential identification of the breast, lung, colon or kidney tissue(s) or breast, lung, colon or kidney cell type(s). For a number of disorders of the above tissues or cells, particularly cancer of the breast, lung, colon and kidney, expression of this gene at significantly higher or lower levels is detected in breast or lung or colon tissues or kidney tissues and cell types (e.g. breast cancer or lung cancer or colon cancer or kidney cancer) or bodily fluids comprising breast, lung, colon or kidney tissue or cells relative to the standard gene expression level, i.e., the expression level in healthy tissue or bodily fluid from an individual not having cancer.

The tissue distribution indicates that polynucleotides and polypeptides corresponding to these newly identified genes expressed in cancerous breast, lung, colon or kidney tissue are useful for the treatment/diagnosis of breast, lung, colon or kidney cancer depending on whether the sequence (listed in Table 1 and Tables 2 through 9 in Example 6) is expressed in cancerous tissue of breast, lung, colon or kidney origin. These proteins identified above may also be involved in apoptosis or tissue differentiation and could accordingly be useful in cancer therapy. Protein, as well as, antibodies directed against the protein or polypeptide may show utility as a tumor marker and/or immunotherapy targets for the above breast, lung colon and kidney tissues.

The term “polypeptide” refers to a biopolymer compound made up of a single chain of amino acid residues linked by peptide bonds. The term “protein” as used herein may be synonymous with the term “polypeptide” or may refer, in addition, to a complex of two or more polypeptides.

The term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

The term “plasmid” refers to a circular double-stranded (ds) DNA construct used as a cloning vector, and which forms an extrachromosomal self-replicating genetic element in many bacteria and some eukaryotes.

A “transcription regulatory region” or “promoter” refers to nucleic acid sequences that influence and/or promote initiation of transcription. Promoters are typically considered to include regulatory regions, such as enhancer or inducer elements. The promoter will generally be appropriate to the host cell in which the target gene is being expressed. The promoter, together with other transcriptional and translational regulatory nucleic acid sequences (also termed “control sequences”), is necessary to express any given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, “operably linked” elements, e.g., enhancers, do not have to be contiguous. Linking is accomplished by ligation at convenient restriction sites. If such sites do not exist, the synthetic oligonucleotide adaptors or linkers are used in accordance with conventional practice.

The term “gene” means the segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons).

The term “sequence identity” means nucleic acid or amino acid sequence identity in two or more aligned sequences, aligned using a sequence alignment program.

The term “% homology” is used interchangeably herein with the term “% identity” and refers to the level of nucleic acid or amino acid sequence identity between two or more aligned sequences, when aligned using a sequence alignment program. For example, 70% homology means the same thing as 70% sequence identity determined by a defined algorithm, and accordingly a homologue of a given sequence has greater than 80% sequence identity over a length of the given sequence. Exemplary levels of sequence identity include, but are not limited to, 80, 85, 90 or 95% or more sequence identity to a given sequence, e.g., the coding sequence for lactoferrin, as described herein.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at “www.ncbi.nlm.gov/BLAST/”. See, also, Altschul, S. F. et al., 1990 and Altschul, S. F. et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences which have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below.the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more. sequence identity with the probe.

Moderate and high stringency hybridization conditions are well known in the art (see, for example, Sambrook et al, 1989, Chapters 9 and 11, and in Ausubel et a., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 233 SSC and 0.5% SDS at room temperature and two additional times in 0.1 X SSC and 0.5% SDS at 42° C.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

Terms “transformed”, “stably transformed” or “transgenic” with reference to a cell means the cell has a non-native (heterologous) nucleic acid sequence integrated into its genome which is maintained through two or more generations.

The term “expression” with respect to a protein or peptide refers to the process by which the protein or peptide is produced based on the nucleic acid sequence of a gene. The process includes both transcription and translation. The term “expression” may also be used with respect to the generation of RNA from a DNA sequence.

The term “introduced” in the context of inserting a nucleic acid sequence into a cell, means “transfection”, or “transformation” or “transduction” and includes the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

By “host cell” is meant a cell which contains a vector and supports the replication, and/or transcription or transcription and translation (expression) of the expression construct. Host cells for use in the present invention can be prokaryotic cells, such as E. coli, or eukaryotic cells such as yeast, plant, insect, amphibian, or mammalian cells. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a polynucleotide of this invention.

The term “purifying” is used interchangeably with the term “isolating” and generally refers to the separation of a particular component from other components of the environment in which it was found or produced.

“Heterologous DNA” or “foreign DNA” refers to DNA which has been introduced into cells from another source, or which is from a source, including the same source, but which is under the control of a promoter or terminator that does not normally regulate expression of the heterologous DNA.

“Heterologous protein” is a protein, including a polypeptide, encoded by a heterologous DNA.

A “signal/targeting/transport sequence” is an N- or C-terminal polypeptide sequence which is effective to localize the polypeptide or protein to which it is attached to a selected intracellular or extracellular region, including an intracellular vacuole or other protein storage body, chloroplast, mitochondria, or endoplasmic reticulum.

A “product” encoded by a DNA molecule includes, for example, RNA molecules and polypeptides.

A DNA sequence is “derived from” a gene if it corresponds in sequence to a segment or region of that gene. Segments of genes which may be derived from a gene include the promoter region, the 5′ untranslated region, and the 3′ untranslated region of the gene.

Therefore, polynucleotides and polypeptides of the invention are useful as reagents for differential identification of the tissue(s) or cell type(s) present in a biological sample and for diagnosis of diseases and conditions which include, but are not limited to, tumors, particularly, those found in the breast, lung, colon or kidney (the tissue type will depend on which polynucleotide or polypeptide is at issue).

Similarly, polypeptides and antibodies directed to these polypeptides are useful in providing immunological probes for differential identification of breast, lung, colon or kidney tissue(s) or cell type(s).

The proteins identified herein may also be involved in apoptosis or tissue differentiation and as such could be useful in breast, lung, colon or kidney cancer therapy, respectively. Protein, as well as, antibodies directed against the protein may show utility as a tumor marker and/or immunotherapy targets for the above listed tissues.

Diagnostic applications for the 385 polynucleotides identified respectively in the file “14dec03.ST25.txt” (generated by PatentIn3.2) attached to this application and incorporated by reference as listed herein in breast, lung, colon, and kidney cancer tissues include that the differentially expressed polynucleotide or polypeptide can be identified in patient tissue in order to identify the presence of a cancer in that patient. Identification of the differentially expressed polynucleotide or polypeptide can be accomplished by standard techniques known in the art for making such identification including, probe hybridization and antibody or ligand binding detection methods. All or part of the polynucleotide or polypeptide can be detected to make a positive identification.

Therapeutic applications for the 385 polynucleotides identified respectively in the file “14dec03.ST25.txt” (generated by PatentIn3.2) attached to this application and incorporated by reference as listed herein in breast, lung, colon, and kidney cancer tissues include blocking the expression of the gene or protein in vivo by administration of an appropriate blocking agent, such as known in the art, for example, but not limited to a polynucleotide (e.g antisense polynucleotide sequence), a ligand binding polypeptide, e.g. such as an antibody or binding portion of an antibody, a peptide, a small organic molecule, and the like, which can bind or interrupt the function of the target polynucleotide or expressed polypeptide molecule. All or part of the polynucleotide or polypeptide can be bound, blocked, arrested or interrupted in its function in the target cancer metabolism.

Table 1 summarizes the information corresponding to each “Unigene No.” (when available) described above and below. Included in the table are the Sequence ID No.s of the particular polynucleotide and the common name of the protein as well as the cancer tissue in which the polynucleotide has been discovered by the inventors as differentially expressed.

The translated amino acid sequence, can be easily translated using known molecular biology techniques. The polypeptides produced by these alternative open reading frames are specifically contemplated by the present invention.

The sequence ID No: and the translated sequence are sufficiently accurate and otherwise suitable for a variety of uses well known in the art and described further below. For instance, the sequence ID NO: is useful for designing nucleic acid hybridization probes that will detect nucleic acid sequences contained in the sequence ID NO:. These probes will also hybridize to nucleic acid molecules in biological samples, thereby enabling a variety of forensic and diagnostic methods of the invention. Similarly, polypeptides identified from translation of the sequences may be used to generate antibodies which bind specifically to the secreted proteins.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

The present invention also relates to the genes corresponding to DNA sequences in the file “14dec03.ST25.txt” (generated by PatentIn3.2) attached to this application and incorporated. by reference or peptides and proteins translated from them. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs. Species homologs may be isolated and identified by making suitable probes or primers from the sequences provided herein and screening a suitable nucleic acid source for the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith and Johnson, Gene 67:3140 (1988). Polypeptides of the invention also can be purified from natural or recombinant sources using antibodies of the invention raised against the secreted protein in methods which are well known in the art.

Polynucleotide and Polypeptide Variants

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences translated from the file “14dec03.ST25.txt” (generated by PatentIn3.2) attached to this application and incorporated by reference can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=I, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C- terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other manual corrections are to be made for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g. to optimize codon expression for a particular host (change codons in the human mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988).) Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310(1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and lie; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

Polynucleotide and Polypeptide Fragments

In the present invention, a “polynucleotide fragment” refers to a short polynucleotide having a nucleic acid sequence contained in the sequence ID NO: The short nucleotide fragments are preferably at least about 15 nt (nt is nucleotides), and more preferably at least about 20 nt, still more preferably at least about 30 nt, and even more preferably, at least about 40 nt in length. A fragment “at least 20 nt in length,” for example, is intended to include 20 or more contiguous bases from the nucleotide sequence shown in the sequence ID NO:. These nucleotide fragments are useful as diagnostic probes and primers as discussed herein. Of course, larger fragments (e.g. 50, 150, 500, 600, 2000 nucleotides) are preferred.

Moreover, representative examples of polynucleotide fragments of the invention, include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050,1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of sequence ID NO:. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in a translation of the sequences in the file “14dec03.ST25.txt” (generated by PatentIn3.2) attached to this application and incorporated by reference. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, or 161 to the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-60, can be deleted from the amino terminus of either the secreted polypeptide or the mature form. Similarly, any number of amino acids, ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of falling within conserved domains are specifically contemplated by the present invention. Moreover, polynucleotide fragments encoding these domains are also contemplated.

Other preferred fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

In the present invention, “epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81:3998-4002, 1983.)

Fragments which function as epitopes may be produced by any conventional means. (See, e.g. Houghten, R. A., Proc. Natl. Acad. Sci. USA 82:5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).)

Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g. in Western blotting.)

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

The invention includes an antibody or binding portion of an antibody that specifically binds a protein found in breast tissue, wherein the protein is an over expressed protein listed herein. Breast cancer diagnosis or prognosis can comprise contacting an antibody or binding portion of an antibody specific to an over expressed protein in breast cancer cells, with the over expressed protein for which the antibody or binding portion thereof is specific. Treatment for breast cancer can comprise blocking an over expressed protein in breast cancer, including those over expressed proteins mentioned herein, with an antibody specific for the over expressed protein.

The invention likewise provides an antibody or binding portion of an antibody that specifically binds a protein found in lung tissue, wherein the protein is an over expressed protein in lung cancer listed herein. Lung cancer diagnosis or prognosis can comprise contacting an antibody or binding portion of an antibody specific to an over expressed protein in lung cancer cells, with the over expressed protein for which the antibody or binding portion thereof is specific. Treatment for lung cancer can comprise blocking an over expressed protein in lung cancer, including those over expressed proteins mentioned herein, with an antibody specific for the over expressed protein.

The invention likewise provides an antibody or binding portion of an antibody that specifically binds a protein found in colon tissue, wherein the protein is an over expressed protein in colon cancer listed herein. Colon cancer diagnosis or prognosis can comprise contacting an antibody or binding portion of an antibody specific to an over expressed protein in colon cancer cells, with the over expressed protein for which the antibody or binding portion thereof is specific. Treatment for colon cancer can comprise blocking an over expressed protein in colon cancer, including those over expressed proteins mentioned herein, with an antibody specific for the over expressed protein.

The invention likewise provides an antibody or binding portion of an antibody that specifically binds a protein found in kidney tissue, wherein the protein is an over expressed protein in kidney cancer listed herein. Kidney cancer diagnosis or prognosis can comprise contacting an antibody or binding portion of an antibody specific to an over expressed protein in kidney cancer cells, with the over expressed protein for which the antibody or binding portion thereof is specific. Treatment for kidney cancer can comprise blocking an over expressed protein in kidney cancer, including those over expressed proteins mentioned herein, with an antibody specific for the over expressed protein.

Manufacture of antibodies can be accomplished by standard antibody techniques known in the art. Diagnosis using antibodies generated to specific proteins or polypeptides can be accomplished, for example, by removing a sample of the tumor tissue or other body material or fluid from the patient and contacting the antibody with the patient's material. Diagnosis standards can be established for each protein and cancer type, as is standardly developed in the art. Treatments can be accomplished after establishing an effective dose of antibody for the particular patient or cancer or protein being targeted. Treatment efficacy can be determined based on any number of parameters including reduction of target protein, reduction of tumor size, or stabilization of disease.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

Moreover, polypeptides of the present invention, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).). Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharnacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hlL-5, have been fused with Fc portions for the purpose of high-throughput screening assays to identify antagonists of hlL-5. (See, D. Bennett et al., J. Molecular Recognition 8:52-58 ,1995; K. Johanson et al., J. Biol. Chem. 270:9459-9471, 1995.)

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).) Thus, any of these above fusions can be engineered using the polynucleotides or the polypeptides of the present invention.

Vectors. Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in the file “14dec03.ST25.txt” (generated by PatentIn3.2) attached to this application and incorporated by reference. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the file “14dec03.ST25.txt” (generated by PatentIn3.2) attached to this application and incorporated by reference will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, “Mendelian Inheritance in Man” available on line through Johns Hopkins University Welch Medical Library). Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of the Polynucleotides in Microarrays

The invention provides a prepared solid support comprising immobilized and separate groups of oligonucleotide or cDNA probes. Each probe group corresponds to a particular region within the reference sequence. The probes can be selected or designed using for example a standard PCR probe selection program such as Probe3 from Massachusetts Institute of Technology (MIT).

The solid phase support can provide an area of about 5 to about 100 square micrometers, on which up to about 100,000 groups of probes can be immobilized in discrete areas according to a predetermined pattern. The prepared solid support can have an associated written or electronic record of the sequence of the probe or probe pairs at any given location on the support, and thus the location on the support of an amplified target can be identified as well. It is understood that a nucleic acid probe of the present invention may contain minor deletions, additions and/or substitutions of nucleic acid bases, to the extent that such alterations do not negatively affect the yield or product obtained to a significant degree.

Oligonucleotide or cDNA probes can include the naturally occurring heterocyclic bases normally found in nucleic acids (uracil, cytosine, thymine, adenine and guanine), as well as modified bases and base analogues. Any modified base or base analogue compatible with hybridization of the probe to a target sequence is useful in the practice of the invention. The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; i.e., can comprise more than one type of base or sugar subunit, and/or the linkages can be of more than one type within the same probe.

The probe can comprise a moiety to facilitate hybridization to its target sequence, as are known in the art, for example, intercalators and/or minor groove binders.

Variations of the bases, sugars, and internucleoside backbone, as well as the presence of any pendant group on the probe, will be compatible with the ability of the probe to bind, in a sequence-specific fashion, with its target sequence. A large number of structural modifications, both known and to be developed, are possible within these bounds. Moreover, synthetic methods for preparing the various heterocyclic bases, sugars, nucleosides and nucleotides which form the probe, and preparation of oligonucleotides of specific predetermined sequence, are well-developed and known in the art. A preferred method for oligonucleotide synthesis incorporates the teaching of U.S. Pat. No. 5,419,966.

Modified oligonucleotides, representing novel sequences and optimized for DNA microarray analysis, are spotted on DNA microarray slides. The microarray slides of the present invention can be of any solid materials and structures suitable for supporting nucleotide hybridization and synthesis. Preferably, the solid phase support comprises at least one substantially rigid surface on which the primers can be immobilized. The solid phase support can be made of, for example, glass, synthetic polymer, plastic, hard non-mesh nylon or ceramic. Other suitable solid support materials are known and readily available to those of skill in the art. The size of the solid support can be any of the standard microarray sizes, useful for DNA microarray technology, and the size may be tailored to fit the particular machine being used to conduct a reaction of the invention. Methods and materials for derivatization of solid phase supports for the purpose of immobilizing oligonucleotides are known to those skill in the art and described in, for example, U.S. Pat. No. 5,919,523, the disclosure of which is incorporated herein by reference.

The oligonucleotide primers of the invention are affixed, immobilized, provided, and/or applied to the surface of the solid support using any available means to fix, immobilize, provide and/or apply the oligonucleotides at a particular location on the solid support. For example, photolithography (Affymetrix, Santa Clara, Calif.) can be used to apply the oligonucleotide primers at particular position on a chip or solid support, as described in the U.S. Pat. Nos. 5,919,523, 5,837,832, 5,831,070, and 5,770,722, which are incorporated herein by reference. The oligonucleotide primers may also be applied to a solid support as described in Brown and Shalon, U.S. Pat. No. 5,807,522 (1998). Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

The target polynucleotides can be detected by using labeled nucleotides (e.g. dNTP-fluorescent label for direct labeling; dNTP-biotin or dNTP-digoxigenin for indirect labeling) incorporated into DNA during the PCR or by other means. For indirectly labeled DNA, the detection may be carried out by fluorescence or other enzyme conjugated streptavidin or anti-digoxigenin antibodies. The PCR method employs detection of the polynucleotides by detecting incorporated label in the newly synthesized complements to the polynucleotide targets. For this purpose, any label that can be incorporated into DNA as it is synthesized can be used, e.g. fluoro-dNTP, biotin-dNTP, or digoxigenin-dNTP, as described above and are known in the art.

Examples of suitable fluorescent labels include fluorescein (FITC), 5,6-carboxymethyl fluorescein, Texas red, nitrobenz-2-oxa-1, 3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 788 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are the preferred form of detection label since they can be directly incorporated during synthesis. Examples of detection labels that can be incorporated into amplified DNA or RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labelling. Cy3.5 and Cy7 are available as avidin or anti-digoxygenin conjugates for secondary detection of biotin- or digoxygenin-labeled probes.

Comparing Differential Expression of Target Polynucleotides from Different Biological Sources.

For most studies involving gene expression, RNA is isolated from specific tissue samples. This RNA is then subjected to reverse transcription using oligo-dT primers and fluorescently labeled dNTPs (usually Cy3 or Cy5 labeled dCTP) resulting in a DNA probe that is fluorescently labeled and has a complementary sequence to the original mRNA. The next step is to hybridize the probe to the immobilized target DNA attached to the microarray. This is done by denaturing the probe with heat or a mild base, to reduce secondary structures that may have formed, and applying it onto the microarray. A cover slip is applied to the array to ensure even distribution of the probe. The array is placed in a warm, humidified chamber overnight to allow the single stranded probe DNA to bind to its complementary single stranded target. The microarray is then removed and washed to remove any nonspecifically bound probe. The arrays are then imaged with a confocal laser scanner. The scanner contains 2 lasers tuned to excite the dye incorporated into the DNA probe and a corresponding filter set to select out excitation emission from the dye (Cy3 or Cy5). The ability to image two fluorescent signals allows for two different RNA samples to be hybridized and directly compared on the same array. This excitation emission signal is recorded via a photomultiplier tube (PMT), digitized, and sent to the computer for later analysis. By examining the intensity of a spot's fluorescence, and the ratio of fluorescence between spots, it possible to determine whether a specific gene is being expressed and the relative expression level of the gene between samples. Other available means for labeling and detecting probes, such as with radioisotopes, enzymes, antibodies, biotin, avidin and like materials known in the art, are within the contemplated means of executing the process.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, or using biotin as a linker to these labels.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc.,1982.)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder.

Moreover, polypeptides of the present invention can be used to treat disease. For example, patients can be administered a polypeptide of the present invention in an effort to replace absent or decreased levels of the polypeptide (e.g. insulin), to supplement absent or decreased levels of a different polypeptide (e.g. hemoglobin S for hemoglobin B), to inhibit the activity of a polypeptide (e.g. an oncogene), to activate a polypeptide (e.g. by binding to a receptor), to reduce the activity of a membrane bound receptor by competing with it for free ligand (e.g. soluble TNF receptors used in reducing inflammation), or to bring about a desired response (e.g. blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Hyperproliferative Disorders

A polypeptide or polynucleotide can be used to treat or detect hyperproliferative disorders, including neoplasms, specifically those found in breast, lung, colon or kidney tissue depending on the polynucleotide or polypeptide being referenced. A polypeptide or polynucleotide of the present invention may inhibit the proliferation of the disorder through direct or indirect interactions. Alternatively, a polypeptide or polynucleotide of the present invention may enhance the proliferation of other cells which can inhibit the hyperproliferative disorder.

For example, by increasing an immune response, particularly increasing antigenic qualities of the hyperproliferative disorder or by proliferating, differentiating, or mobilizing T-cells; hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by a polynucleotide or polypeptide of the present invention include, but are not limited to neoplasms located in the breast, lungs, colon and kidney. Similarly, other hyperproliferative disorders can also be treated or detected by a polynucleotide or polypeptide of the present invention.

Chemotaxis

A polynucleotide or polypeptide of the present invention may have chemotaxis activity. A chemotaxic molecule attracts or mobilizes cells (e.g. monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide of the present invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a polynucleotide or polypeptide of the present invention may inhibit chemotactic activity. These molecules could also be used to treat disorders. Thus, a polynucleotide or polypeptide of the present invention could be used as an inhibitor of chemotaxis.

Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g. receptors), or small molecules.

Preferably, the molecule is closely related to the natural ligand of the polypeptide, e.g. a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which the polypeptide binds, or at least, a fragment of the receptor capable of being bound by the polypeptide (e.g. active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g. biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient by activating or inhibiting the polypeptide/molecule. Moreover, the assays can discover agents which may inhibit or enhance the production of the polypeptide from suitably manipulated cells or tissues.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity and (b) determining if a biological activity of the polypeptide has been altered.

The methods of the invention include using in a diagnostic or therapeutic context an isolated nucleic acid molecule comprising a nucleotide sequence which is at least 95% identical to a sequence of at least about 30 contiguous nucleotides in the nucleotide sequence sequencelD NO: of the file “14dec03.ST25.txt” (generated by PatentIn3.2) attached to this application and incorporated by reference.

The nucleic acid molecule used in the methods can include said sequence of contiguous nucleotides is included in the nucleotide sequence sequencelD NO: of the file “14dec03.ST25.txt” (generated by PatentIn3.2) attached to this application and incorporated by reference in the range of positions beginning with the nucleotide at about the position of the 5′ Nucleotide of the Sequence and ending with the nucleotide at about the position of the 3′ Nucleotide of the Sequence as defined for the file “14dec03.ST25.txt” (generated by PatentIn3.2) attached to this application and incorporated by reference.

Any nucleic acid molecule for the inventive methods can have a sequence of contiguous nucleotides is included in the nucleotide sequence sequence ID NO: of the file “14dec03.ST25.txt” (generated by PatentIn3.2) attached to this application and incorporated by reference in the range of positions beginning with the nucleotide at about the position of the 5′ Nucleotide of the Start Codon and ending with the nucleotide at about the position of the 3′ Nucleotide of the Sequence as defined for sequences in the file “14dec03.ST25.txt” (generated by PatentIn3.2) attached to this application and incorporated by reference.

A nucleic acid molecule can have a sequence of contiguous nucleotides included in the nucleotide sequence of sequencelD NO: of the file “14dec03.ST25.txt” (generated by PatentIn3.2) attached to this application and incorporated by reference in the range of positions beginning with the nucleotide at about the position of the 5′ Nucleotide of the First Amino Acid and ending with the nucleotide at about the position of the 3′ Nucleotide of the Sequence as defined for sequences in the file “14dec03.ST25.txt” (generated by PatentIn3.2) attached to this application and incorporated by reference.

An isolated nucleic acid molecule useful in the methods of the invention can comprise a nucleotide sequence which is at least 95% identical to a sequence of at least about 150 contiguous nucleotides in the nucleotide sequence of the file “14dec03.ST25.txt” (generated by PatentIn3.2) attached to this application and incorporated by reference.

Such an isolated nucleic acid molecule can comprise a nucleotide sequence which is at least 95% identical to a sequence of at least about 500 contiguous nucleotides in the nucleotide sequence of the file “14dec03.ST25.txt” (generated by PatentIn3.2) attached to this application and incorporated by reference.

A nucleic acid molecule in the methods can comprise a nucleotide sequence which is at least 95% identical to the nucleotide sequence of the file “14dec03.ST25.txt” (generated by PatentIn3.2) attached to this application and incorporated by reference beginning with the nucleotide at about the position of the 5′ Nucleotide of the First Amino Acid and ending with the nucleotide at about the position of the 3′ Nucleotide of the Sequence as defined for sequences in the file “14dec03.ST25.txt” (generated by PatentIn3.2) attached to this application and incorporated by reference.

Use of an isolated nucleic acid molecule comprising a nucleotide sequence which is at least 95% identical to the complete nucleotide sequence of the file “14dec03.ST25.txt” (generated by PatentIn3.2) attached to this application and incorporated by reference is also contemplated.

An isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only A residues or of only T residues, is likewise useful with the herein described methods.

An isolated nucleic acid molecule comprising a nucleotide sequence which is at least 95% identical to a sequence of at least 30 contiguous nucleotides in the nucleotide sequence of a human sequence identified in the file “14dec03.ST25.txt” (generated by PatentIn3.2) attached to this application and incorporated by reference, which DNA molecule is contemplated in the methods of the invention.

An isolated nucleic acid molecule for the inventive methods can also include wherein said sequence of at least 30 contiguous nucleotides is included in the nucleotide sequence of the complete open reading frame sequence

An isolated nucleic acid molecule for the inventive methods can also include comprising a nucleotide sequence which is at least 95% identical to sequence of at least 150 contiguous nucleotides in the nucleotide sequence

An isolated nucleic acid molecule for the inventive methods can also include comprising a nucleotide sequence which is at least 95% identical to sequence of at least 500 contiguous nucleotides in the nucleotide sequence encoded by said human cDNA clone.

An isolated nucleic acid molecule for the inventive methods can also include comprising a nucleotide sequence which is at least 95% identical to the complete nucleotide sequence encoded by said human cDNA clone.

Another inventive method includes a method for detecting in a biological sample a nucleic acid molecule comprising a nucleotide sequence which.is at least 95% identical to a sequence of at least 30 contiguous nucleotides in a sequence selected from the group consisting of: a nucleotide sequence of sequences defined in the file “14dec03.ST25.txt” (generated by PatentIn3.2) attached to this application and incorporated by reference; which method comprises a step of comparing a nucleotide sequence of at least one nucleic acid molecule in said sample with a sequence selected from said group and determining whether the sequence of said nucleic acid molecule in said sample is at least 95% identical to said selected sequence.

The above method can include a step of comparing sequences comprising determining the extent of nucleic acid hybridization between nucleic acid molecules in said sample and a nucleic acid molecule comprising said sequence selected from said group. Similarly, a step of comparing sequences is performed by comparing the nucleotide sequence determined from a nucleic acid molecule in said sample with said sequence selected from said group. The nucleic acid molecules can comprise DNA molecules or RNA molecules.

A method for identifying the species, tissue or cell type of a biological sample is also contemplated which method comprises a step of detecting nucleic acid molecules in said sample, if any, comprising a nucleotide sequence that is at least 95% identical to a sequence of at least 30 contiguous nucleotides in a sequence selected from the group consisting of: a nucleotide sequence in the file “14dec03.ST25.txt” (generated by PatentIn3.2) attached to this application and incorporated by reference.

The method for identifying the species, tissue or cell type of a biological sample can comprise a step of detecting nucleic acid molecules comprising a nucleotide sequence in a panel of at least two nucleotide sequences, wherein at least one sequence in said panel is at least 95% identical to a sequence of at least 30 contiguous nucleotides in a sequence selected from said group.

The invention includes a method for diagnosing in a subject a pathological condition associated with abnormal structure or expression of a gene encoding a secreted protein or membrane-bound protein translated from the sequences identified in the file “14dec03.ST25.txt” (generated by PatentIn3.2) attached to this application and incorporated by reference, which method comprises a step of detecting in a biological sample obtained from said subject nucleic acid molecules, if any, comprising a nucleotide sequence that is at least 95% identical to a sequence of at least 30 contiguous nucleotides in a sequence selected from the group consisting of: a nucleotide sequence of the file “14dec03.ST25.txt” (generated by PatentIn3.2) attached to this application and incorporated by reference.

The method for diagnosing a pathological condition (e.g. breast, lung, colon or kidney cancer) can comprise a step of detecting nucleic acid molecules comprising a nucleotide sequence in a panel of at least two nucleotide sequences, wherein at least one sequence in said panel is at least 95% identical to a sequence of at least 30 contiguous nucleotides in a sequence selected from said group.

In furtherance of the methods of the invention, an isolated antibody which binds specifically to a polypeptide comprising an amino acid sequence that is at least 90% identical to a sequence of at least 10 contiguous amino acids in a sequence selected from the group consisting of: an amino acid sequence translated from sequences defined in the file “14dec03.ST25.txt” (generated by PatentIn3.2) attached to this application and incorporated by reference is useful as a reagent in a diagnostic or therapeutic procedure.

The invention further contemplates a method for detecting in a biological sample a polypeptide comprising an amino acid sequence which is at least 90% identical to a sequence of at least 10 contiguous amino acids in a sequence selected from the group consisting of: an amino acid sequence of translated sequences defined in the file “14dec03.ST25.txt” (generated by PatentIn3.2) attached to this application and incorporated by reference which method comprises a step of comparing an amino acid sequence of at least one polypeptide molecule in said sample with a sequence selected from said group and determining whether the sequence of said polypeptide molecule in said sample is at least 90% identical to said sequence of at least 10 contiguous amino acids.

A method of the invention can include a method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence that is at least 90% identical to a sequence of at least 10 contiguous amino acids in a sequence selected from the group consisting of: an amino acid sequence of translated from sequences as defined in the file “14dec03.ST25.txt” (generated by PatentIn3.2) attached to this application and incorporated by reference.

The method can include wherein said step of comparing sequences is performed by comparing the amino acid sequence determined from a polypeptide molecule in said sample with said sequence selected from said group.

A method for identifying the species, tissue or cell type of a biological sample which method comprises a step of detecting polypeptide molecules in said sample, if any, comprising an amino acid sequence that is at least 90% identical to a sequence of at least 10 contiguous amino acids in a sequence selected from the group consisting of: an amino acid sequence translated from sequences as defined in the file “14dec03.ST25.txt” (generated by PatentIn3.2) attached to this application and incorporated by reference is also contemplated.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel is at least 90% identical to a sequence of at least 10 contiguous, amino acids in a sequence selected from the above group.

Also preferred is a method for diagnosing in a subject a pathological condition associated with abnormal structure or expression of a gene encoding a secreted protein or membrane-bound protein identified in the file “14dec03.ST25.txt” (generated by PatentIn3.2) attached to this application and incorporated by reference, which method comprises a step of detecting in a biological sample obtained from said subject polypeptide molecules comprising an amino acid sequence in a panel of at least two amino acid sequences, wherein at least one sequence in said panel is at least 90% identical to a sequence of at least 10 contiguous amino acids in a sequence selected from the group consisting of: an amino acid sequence of translated from sequences as defined in the file “14dec03.ST25.txt” (generated by PatentIn3.2) attached to this application and incorporated by reference.

In any of these methods, the step of detecting said polypeptide molecules includes using an antibody.

Also preferred for use in the methods of the invention is an isolated nucleic acid molecule comprising a nucleotide sequence which is at least 95% identical to a nucleotide sequence encoding a polypeptide wherein said polypeptide comprises an amino acid sequence that is at least 90% identical to a sequence of at least 10 contiguous amino acids in a sequence selected from the group consisting of: an amino acid sequence of translated from sequences as defined in the file “14dec03.ST25.txt” (generated by PatentIn3.2) attached to this application and incorporated by reference.

Also preferred for use in the methods of the invention is an isolated nucleic acid molecule, wherein said nucleotide sequence encoding a polypeptide has been optimized for expression of said polypeptide in a prokaryotic host.

Also preferred for use in the methods of the invention is an isolated nucleic acid molecule, wherein said polypeptide comprises an amino acid sequence selected from the group consisting of: an amino acid sequence of translated from sequences as defined in the file “14dec03.ST25.txt” (generated by PatentIn3.2) attached to this application and incorporated by reference.

Further preferred is a method of making a recombinant vector comprising inserting any of the above isolated nucleic acid molecule into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a secreted portion of a human secreted protein comprising an amino acid sequence selected from the group consisting of: an amino acid sequence translated from sequences in the file “14dec03.ST25.txt” (generated by PatentIn3.2) attached to this application and incorporated by reference and said position of the First Amino Acid of the Secreted Portion translated from sequences is defined in the file “14dec03.ST25.txt” (generated by PatentIn3.2) attached to this application and incorporated by reference. The isolated polypeptide produced by this method is also preferred.

Having generally described the invention, the same will be more readily understood by reference to the following examples, which are provided by way of illustration and are not intended as limiting. 2. EXAMPLES

The following examples are put forth so as to provide those of ordinary skill in the art with a compete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts of weight, molecular weight is weight average molecular weight, temperature is in degrees Celsius, and pressure is at or near atmospheric.

### Example 1

### Tissue Distribution of Polypeptide in Breast, Lung, Colon or Kidney Cancer Tissue

Tissue distribution of mRNA expression of polynucleotides of the present invention is determined using protocols for Northern blot analysis, described by, among others, Sambrook et al. For example, a cDNA probe produced by the method described in Example 1 is labeled with p³² using the rediprime.™ DNA labeling system (Amersham Life Science), according to manufacturer's instructions. After labeling, the probe is purified using CHROMA SPIN-100.™ column (Clontech Laboratories, Inc.), according to manufacturer's protocol number PT 1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70° C. overnight, and the films developed according to standard procedures.

### Example 2

### Chromosomal Mapping of the Polynucleotides

An oligonucleotide primer set is designed according to the sequence at the 5′ end of the sequences in the file “14dec03.ST25.txt” (generated by PatentIn3.2) attached to this application and incorporated by reference. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C.

Human, mouse, and hamster DNA is used as template in addition to a somatic cell hybrid panel containing individual chromosomes or chromosome fragments (Bios, Inc). The reaction is analyzed on either 8% polyacrylamide gels or 3.5% agarose gels. Chromosome mapping is determined by the presence of an approximately 100 bp PCR fragment in the particular somatic cell hybrid.

### Example 3

### Expression of a Polypeptide in Mammalian Cells

The polypeptide for use in the methods of the present invention can be expressed in a mammalian cell. A typical mammalian expression vector contains a promoter element, which mediates the initiation of transcription of MRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from retroviruses, e.g. RSV, HTLVI, HIVI and the early promoter of the cytomegalovirus (CMV). However, cellular elements can also be used (e.g. the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, COS-1, COS-7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful in developing cell lines that carry several hundred or even several thousand copies of the gene of interest. (See, e.g. Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g. with the restriction enzyme cleavage sites BamHl, Xbal and Asp718, facilitate the cloning of the gene of interest. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

A polynucleotide of the present invention is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence. (See, e.g. WO 96/34891.)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five micrograms (μg) of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 micromolar (μM), 2 μgM, 5 μM, 10 μM, 20 μM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Production of an Antibody from a Polypeptide

Antibodies for use in the methods of the present invention can be prepared by a variety of methods. (See, Current Protocols, Chapter 2.) For example, cells expressing a polypeptide of the present invention are administered to an animal to induce the production of sera containing polyclonal antibodies. In a preferred method, a preparation of the secreted or membrane-bound protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Kohler et al., Eur. J. Immunol. 6:511 (1976); Kohler et al., Eur. J. lmmunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 5

### Method of Determining Alterations in a Gene Corresponding to a Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in sequences in the file “14dec03.ST25.txt” (generated by PatentIn3.2) attached to this application and incorporated by reference. Suggested PCR conditions consist of 35 cycles at 95° C. for 30 seconds; 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons are also determined and genomic PCR products analyzed to confirm the results. PCR products harboring suspected mutations is then cloned and sequenced to validate the results of the direct sequencing.

PCR products is cloned into T-tailed vectors as described in Holton, T. A. and Graham, M. W., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diarnino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 6

### Detection of Levels of mRNA Expressed in Diseased Tissues Using Arrays

(Supplemental data supplied in the accompanying Excel spreadsheet show the results used to support this patent by identifying the differential expression associated with diseased verusus cancerous tissue.)

The identified sequences representing a variety of candidate genes to be screened for differential expression in cancer. 5′-modified oligonucleotides representing the novel sequences and optimized for DNA microarray analysis can be designed using a combination of GENSCAN, Pfam, Primer3 (release 0.9 (1998) MIT Whitehead Institute) and spotted onto reflective glass slides (Amersham) according to methods well known in the art. These microarrays can then be used to study differential gene expression in selected cell lines and patient tissues. Normal tissues and tissues from cancer patients were processed to generate T7 RNA polymerase transcribed polynucleotides, which were, in turn, assessed for expression in the microarrays.

The microarrays were then used to study differential gene expression in various disease samples and appropriate normal control samples. Target polynucleotides were prepared from total RNA, first reverse transcribed into codon using a primer containing a T7 RNA polymerase promoter, followed by second strand DNA synthesis. The codon was then transcribed in vitro to produce antisense RNA using the T7 promoter-mediated expression (see, e.g., Luo et al. (1999) Nature Med 5:117-122). In one embodiment, biotin labeled antisense RNA is generated from the RNA isolated from sample and control tissue by standard protocols and hybridized to the immobilized oligonucleotides representing the novel cancer genes.

Hybridization is detected by binding the biotinylated antibody with streptavidin which is in turn bound by a first anti-streptavidin antibody and the signals are amplified using a secondary antibody which binds the anti-streptavidin antibody and is conjugated to the fluorescent label Cy3. The fluorescent label is detected by commercially available array scanners. These indirect immunofluorescence techniques are known in the art.

The array images generated by hybridization were captured by a laser array scanner (e.g., GenePix 4000B by Axon Instruments, Foster City, Calif.) and the data was analyzed using DNA array image analysis software (ImaGene 4.0, Biodiscovery, Inc.) and an in-house microarray data analysis software (Mergen).

In one embodiment, the procedure provides for fluorescent labeling of RNA. Probes are labeled by making fluorescently labeled codon from the RNA starting material. Fluorescently labeled cDNAs prepared from the diseased RNA sample are compared to fluorescently labeled cDNAs prepared from normal cell RNA sample. For example, in one embodiment the codon probes from the normal cells are labeled with Cy3 fluorescent dye (green) and the codon probes prepared from the diseased cells are labeled with Cy5 fluorescent dye (red).

The differential expression assay can be performed by mixing equal amounts of probes from disease cells and normal cells of the same patient. The arrays were prehybridized by incubation for about 2 hrs at 60° C. in 5×SSC, 0.2% SDS, 1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS). After hybridization, the array was washed at 55° C. three times as follows: (i) first wash in 1×SSC, 0.2% SDS; (ii) second wash in 0.1×SSC, 0.2% SDS; and (iii) third wash in 0.1×SSC.

The arrays were then scanned for green and/or red fluorescence, the images were processed using the AutoGene Microarray Image Analysis System™ (BioDiscovery, Inc., Los Angeles, Calif.) and the data from each scan set was normalized to produce the data in Tables 2 through 9.

### Results of Example 6

TABLE 2
Genes Over Expressed in Breast Tumors (part one)

| | Control | Cancer | Control | Cancer | Control | Cancer | Control | Cancer | Control | Cancer |
|---|---|---|---|---|---|---|---|---|---|---|
| | Patient 0005 | | Patient 01010416 | | Patient 01010417 | | Patient 01010421 | | Patient 01010425 | |
| Mergen ID | Control | Cancer | Control | Cancer | Control | Cancer | Control | Cancer | Control | Cancer |
| Clone-01 | 5770 | 4268 | 3141 | 14219 | 2923 | 6151 | 967 | 1777 | 2392 | 10520 |
| Clone-02 | 207 | 4311 | 1431 | 5814 | 1294 | 9171 | 181 | 1852 | 363 | 17012 |
| Clone-03 | 5353 | 20197 | 25654 | 64865 | 2185 | 41141 | 1346 | 930 | 7412 | 63341 |
| Clone-04 | 662 | 3432 | 649 | 4246 | 1083 | 8934 | 223 | 5917 | 939 | 6966 |
| Clone-06 | 182 | 581 | 292 | 1129 | 734 | 1613 | 95 | 1280 | 342 | 3073 |
| Clone-07 | 1558.5 | 6587.5 | 1630.1 | 7862.5 | 1155.6 | 4709.0 | 929.7 | 3721.1 | 2692.3 | 18362.3 |
| Clone-08 | 512.3 | 1713.6 | 550.1 | 495.3 | 523.6 | 1650.3 | 251.3 | 1952.8 | 678.3 | 2336.5 |
| Clone-09 | 91.1 | 557.1 | 131.4 | 1778.8 | 297.9 | 4212.9 | 103.4 | 6222.5 | 199.4 | 130.0 |
| Clone-10 | 170.7 | 2861.4 | 1737.9 | 7657.5 | 644.2 | 26411.9 | 111.4 | 29855.3 | 197.1 | 116.4 |
| Clone-11 | 145.8 | 412.6 | 213.1 | 839.3 | 50.6 | 4523.1 | 11.0 | 4537.4 | 31.0 | 11.0 |
| Clone-12 | 4300.7 | 21092.8 | 5972.5 | 19377.7 | 1350.6 | 8406.6 | 1269.9 | 10245.8 | 8879.7 | 27723.5 |
| Clone-15 | 340.0 | 1525.8 | 640.9 | 2494.4 | 368.3 | 758.7 | 137.6 | 768.7 | 900.6 | 4722.0 |
| Clone-16 | 638.8 | 4956.6 | 1926.9 | 1234.4 | 2062.7 | 7584.7 | 411.2 | 4341.9 | 1189.5 | 5918.7 |
| Clone-17 | 210.7 | 2391.7 | 558.6 | 7158.5 | 180.2 | 1727.0 | 122.1 | 365.9 | 1288.9 | 5158.1 |
| Clone-18 | 6578.1 | 34061.3 | 11788.7 | 40709.0 | 2568.4 | 9246.5 | 1580.0 | 7390.2 | 31575.8 | 38034.5 |
| Clone-19 | 283.2 | 2625.5 | 526.7 | 620.8 | 496.6 | 3265.8 | 176.9 | 4360.6 | 259.8 | 1054.6 |
| Clone-20 | 256.0 | 6112.7 | 363.0 | 142.3 | 533.2 | 5259.8 | 185.1 | 729.7 | 373.7 | 74.8 |
| Clone-21 | 779.0 | 3076.4 | 699.3 | 1234.8 | 682.3 | 1684.5 | 299.8 | 1042.3 | 638.5 | 4124.3 |
| Clone-22 | 818.8 | 10746.6 | 1080.1 | 4329.6 | 323.9 | 1143.0 | 191.0 | 724.9 | 3919.2 | 6118.3 |
| Clone-23 | 215.2 | 1695.6 | 764.8 | 774.5 | 377.7 | 846.5 | 171.4 | 989.6 | 467.3 | 2961.8 |
| Clone-24 | 253.4 | 822.7 | 157.8 | 1045.6 | 181.8 | 1041.3 | 88.9 | 358.8 | 273.1 | 3974.6 |
| Clone-25 | 1709.6 | 6666.2 | 2064.9 | 4322.8 | 2526.9 | 7727.1 | 517.6 | 28241.0 | 1230.5 | 29391.3 |
| Clone-26 | 419.2 | 1352.6 | 831.5 | 1876.9 | 200.3 | 2323.6 | 248.5 | 964.0 | 551.2 | 2745.1 |
| Clone-27 | 509.6 | 1121.8 | 663.5 | 2065.0 | 975.0 | 961.0 | 213.0 | 1188.8 | 542.0 | 2664.4 |

TABLE 3
Genes Over Expressed in Breast Tumors (part two)

| | Control | Cancer | Control | Cancer | Control | Cancer | Control | Cancer | Control | Cancer |
|---|---|---|---|---|---|---|---|---|---|---|
| | Patient 01010437 | | Patient 01010438 | | Patient 01010443 | | Patient P02003 | | Patient 01010427 | |
| Mergen ID | Control | Cancer | Control | Cancer | Control | Cancer | Control | Cancer | Control | Cancer |
| Clane-01 | 1181 | 5027 | 1207 | 5544 | 1520 | 3293 | 1656 | 6863 | | |
| Clone-02 | 398 | 3042 | 313 | 3718 | 1402 | 2580 | 550 | 909 | | |
| Clone-03 | 2782 | 1627 | 1872 | 6444 | 3779 | 28723 | 2531 | 22190 | | |
| Clone-04 | 728 | 4706 | 561 | 3906 | 1339 | 3900 | 347 | 1215 | | |
| Clone-06 | 234 | 644 | 391 | 1119 | 499 | 1442 | 121 | 337 | | |
| Clone-07 | 2154.6 | 17568.3 | 1880.9 | 8399.3 | 1446.5 | 9486.2 | 1263.3 | 1073.2 | 358.0 | 5949.0 |
| Clone-08 | 675.3 | 852.0 | 203.5 | 707.5 | 454.4 | 2802.4 | 410.9 | 633.8 | 208.8 | 1755.5 |
| Clane-09 | 89.7 | 1021.5 | 141.7 | 134.5 | 321.5 | 11233.0 | 145.5 | 1448.8 | 561.5 | 5794.0 |
| Clone-10 | 261.3 | 5229.9 | 115.2 | 174.6 | 427.4 | 44799.2 | 257.5 | 17014.2 | 361.6 | 38624.8 |
| Clone-11 | 73.8 | 643.3 | 15.7 | 26.8 | 103.5 | 12604.7 | 11.0 | 1765.4 | 26.3 | 1484.4 |
| Clone-12 | 3663.4 | 20477.8 | 862.3 | 9924.2 | 1901.1 | 6474.9 | 7059.4 | 12870.4 | 855.0 | 16149.9 |
| Clone-15 | 457.6 | 1914.4 | 287.0 | 1350.9 | 345.9 | 693.2 | 898.2 | 1094.3 | 200.1 | 660.9 |
| Clone-16 | 1298.3 | 1448.5 | 521.4 | 7112.5 | 476.2 | 707.7 | 973.4 | 9765.9 | 625.8 | 3800.7 |
| Clone-17 | 570.4 | 1798.1 | 207.0 | 748.7 | 219.2 | 995.2 | 465.8 | 9327.7 | 84.3 | 477.3 |
| Clone-18 | 10578.1 | 43845.9 | 1293.5 | 4978.1 | 2645.0 | 9846.2 | 16085.8 | 19792.9 | 1381.0 | 7508.4 |
| Clone-19 | 852.0 | 3590.2 | 235.3 | 1060.8 | 341.1 | 2422.6 | 467.2 | 2766.9 | 131.3 | 3780.7 |
| Clone-20 | 307.4 | 1537.0 | 231.3 | 2492.8 | 918.6 | 8903.8 | 484.6 | 5071.5 | 118.8 | 708.0 |
| Clone-21 | 990.4 | 6043.7 | 525.4 | 1744.2 | 402.3 | 1210.6 | 552.8 | 788.6 | 240.5 | 932.7 |
| Clone-22 | 1119.2 | 7032.0 | 363.7 | 4297.0 | 799.5 | 1113.9 | 4708.9 | 6186.2 | 161.7 | 1042.8 |
| Clone-23 | 415.9 | 2005.3 | 162.3 | 560.3 | 294.2 | 1220.2 | 205.7 | 1339.8 | 164.6 | 789.8 |
| Clone-24 | 285.7 | 933.2 | 170.1 | 1237.4 | 173.9 | 1204.3 | 120.6 | 251.3 | 34.1 | 322.0 |
| Clone-25 | 902.3 | 58795.4 | 859.8 | 4436.0 | 2479.4 | 15199.0 | 687.4 | 2386.4 | 289.8 | 30159.8 |
| Clone-26 | 377.2 | 1141.0 | 309.0 | 1033.1 | 325.3 | 1702.6 | 446.3 | 1002.4 | 265.8 | 1209.9 |
| Clone-27 | 616.8 | 2232.2 | 405.5 | 1494.5 | 714.1 | 3321.0 | 724.4 | 406.9 | 215.4 | 1045.7 |

TABLE 4
Genes Over Expressed in Colon Tumors (part one)

| | Control | Cancer | Control | Cancer | Control | Cancer | Control | Cancer | Control | Cancer |
|---|---|---|---|---|---|---|---|---|---|---|
| | Patient 01010023 | | Patient 01010126 | | Patient 01010150 | | Patient 01010169 | | Patient 01010078 | |
| Mergen ID | Control | Cancer | Control | Cancer | Control | Cancer | Control | Cancer | Control | Cancer |
| Clone-13 | 35.7 | 149.0 | 20.8 | 1378.2 | 14.9 | 192.0 | 13.0 | 1027.5 | 56.9 | 40.8 |
| Clone-14 | 15.0 | 43.5 | 32.5 | 163.4 | 23.8 | 261.2 | 1.1 | 103.0 | 37.5 | 416.7 |
| Clone-32 | 1528 | 14970 | 307 | 4303 | 864 | 4327 | 999 | 2376 | 380 | 2304 |
| Clone-33 | 5109 | 21561 | 1671 | 17031 | 1145 | 31962 | 2689 | 8080 | 2173 | 29803 |
| Clone-34 | 592 | 2156 | 341 | 1047 | 797 | 1721 | 247 | 3855 | 231 | 3260 |
| Clone-35 | 215 | 20194 | 676 | 8372 | 214 | 42265 | 87 | 4502 | 1479 | 11747 |
| Clone-36 | 993 | 4366 | 1618 | 12834 | 3958 | 9257 | 4013 | 14186 | 575 | 7026 |
| Clone-37 | 558 | 486 | 243 | 850 | 168 | 1284 | 159 | 1635 | 93 | 1146 |
| Clone-38 | 114 | 1425 | 79 | 185 | 311 | 608 | 168 | 154 | 35 | 627 |
| Clone-39 | 405 | 8634 | 247 | 15211 | 470 | 7174 | 1518 | 11661 | 881 | 11548 |
| Clone-40 | 153 | 1180 | 17 | 403 | 73 | 5218 | 91 | 941 | 184 | 965 |

TABLE 5
Genes Over Expressed in Colon Tumors (part two)

| | Control | Cancer | Control | Cancer | Control | Cancer | Control | Cancer | Control | Cancer |
|---|---|---|---|---|---|---|---|---|---|---|
| | Patient 01020191 | | Patient 1010191 | | Patient 1010402 | | Patient 1010413 | | Patient 1020025 | |
| Mergen ID | Control | Cancer | Control | Cancer | Control | Cancer | Control | Cancer | Control | Cancer |
| Clone-13 | 24.2 | 104.0 | 4 | 361 | 21 | 37 | 37 | 48 | | |
| Clone-14 | 17.0 | 189.1 | 8 | 413 | 1 | 39 | 10 | 450 | | |
| Clone-32 | 2360 | 8741 | 369 | 2810 | 515 | 2132 | 423 | 1228 | 766 | 9147 |
| Clone-33 | 2501 | 14886 | 3793 | 16131 | 5474 | 14254 | 1539 | 21185 | 4229 | 10946 |
| Clone-34 | 384 | 6983 | 1236 | 7031 | 563 | 11480 | 444 | 3715 | 262 | 1529 |
| Clone-35 | 113 | 16344 | 225 | 32480 | 115 | 2695 | 892 | 5215 | 526 | 3046 |
| Clone-36 | 1365 | 9727 | 6814 | 12528 | 3562 | 14063 | 841 | 5291 | 2137 | 7309 |
| Clone-37 | 331 | 3035 | 412 | 1189 | 605 | 2056 | 340 | 2463 | 173 | 1447 |
| Clone-38 | 42 | 521 | 52 | 1688 | 225 | 6132 | 96 | 448 | 90 | 329 |
| Clone-39 | 489 | 2776 | 719 | 5292 | 754 | 4812 | 512 | 1971 | 238 | 9650 |
| Clone-40 | 931 | 1702 | 69 | 1563 | 415 | 833 | 79 | 2755 | 72 | 536 |

TABLE 6
Genes Over Expressed in Lung Tumors (part one)

| | Control | Cancer | Control | Cancer | Control | Cancer | Control | Cancer | Control | Cancer |
|---|---|---|---|---|---|---|---|---|---|---|
| | Patient 01010123 | | Patient 01010400 | | Patient 01010411 | | Patient 01010424 | | Patient 01010429 | |
| Mergen ID | Control | Cancer | Control | Cancer | Control | Cancer | Control | Cancer | Control | Cancer |
| Clone-28 | 6804 | 21461 | 1561 | 3574 | 1570 | 6163 | 4524 | 13950 | 5395 | 12270 |
| Clone-29 | 7065.6 | 7285.9 | 161.3 | 3024.5 | 120.0 | 1101.2 | 691.8 | 9283.7 | 1201.5 | 7471.9 |
| Clone-30 | 1872.8 | 3357.1 | 177.4 | 1238.2 | 76.4 | 930.9 | 16.0 | 192.4 | 276.0 | 2905.9 |
| Clone-31 | 4356.7 | 4761.6 | 324.5 | 1485.9 | 110.4 | 1969.0 | 188.9 | 929.2 | 583.1 | 7791.4 |

TABLE 7
Genes Over Expressed in Lung Tumors (part two)

| | Control | Cancer | Control | Cancer | Control | Cancer | Control | Cancer | Control | Cancer |
|---|---|---|---|---|---|---|---|---|---|---|
| | Patient 01010432 | | Patient 01010433 | | Patient 01010435 | | Patient 01010448 | | Patient 01010449 | |
| Mergen ID | Control | Cancer | Control | Cancer | Control | Cancer | Control | Cancer | Control | Cancer |
| Clone-28 | 3158 | 8448 | 6897 | 22259 | 4304 | 19003 | 1599 | 7369 | 2227 | 10710 |
| Clone-29 | 14.4 | 78.8 | 291.6 | 2165.8 | 81.0 | 1071.4 | 6824.9 | 5906.2 | 1311.5 | 3229.2 |
| Clone-30 | 37.3 | 194.6 | 329.9 | 3080.0 | 18.5 | 79.0 | 1596.7 | 270.8 | 483.0 | 1621.4 |
| Clone-31 | 126.0 | 433.2 | 351.8 | 1293.8 | 73.7 | 820.2 | 1170.8 | 1963.1 | 837.4 | 1491.8 |

TABLE 8
Genes Over Expressed in Kidney Tumors (part one)

| | Control | Cancer | Control | Cancer | Control | Cancer | Control | Cancer | Control | Cancer |
|---|---|---|---|---|---|---|---|---|---|---|
| | Patient 01020012 | | Patient 01020026 | | Patient 01020027 | | Patient 01020029 | | Patient 01020068 | |
| Mergen ID | Control | Cancer | Control | Cancer | Control | Cancer | Control | Cancer | Control | Cancer |
| Clone-41 | 190 | 8608 | 153 | 1980 | 182 | 1388 | 404 | 2682 | 217 | 9741 |
| Clone-42 | 566 | 5497 | 141 | 1498 | 206 | 1158 | 529 | 7310 | 415 | 3486 |
| Clone-43 | 76 | 1131 | 133 | 306 | 63 | 2166 | 206 | 1511 | 228 | 852 |
| Clone-44 | 241 | 5963 | 74 | 522 | 59 | 2762 | 126 | 7584 | 283 | 30938 |
| Clone-45 | 2184.3 | 13265.3 | 358.7 | 2736.7 | 327.4 | 1863.3 | 955.2 | 9024.1 | 1546.0 | 12236.8 |
| Clone-46 | 962.5 | 4314.5 | 298.2 | 1189.9 | 258.3 | 1339.7 | 525.8 | 2699.9 | 527.7 | 1467.1 |
| Clone-47 | 1494.8 | 9989.7 | 444.7 | 3529.0 | 278.4 | 2961.6 | 1488.9 | 11933.3 | 575.6 | 14658.8 |
| Clone-48 | 1074.4 | 5027.8 | 78.8 | 376.7 | 20.3 | 93.8 | 183.9 | 767.9 | 375.9 | 2620.4 |
| Clone-49 | 4329.7 | 58342.6 | 1008.9 | 27846.2 | 1020.5 | 978.9 | 1944.5 | 21463.2 | 2351.5 | 58253.9 |
| Clone-50 | 624.6 | 7496.5 | 267.0 | 2181.3 | 107.1 | 333.3 | 176.4 | 3424.0 | 619.9 | 8192.7 |
| Clone-51 | 2886.0 | 13318.2 | 546.7 | 2293.5 | 533.5 | 2477.3 | 934.8 | 8117.2 | 1912.7 | 10248.6 |
| Clone-52 | 1653.5 | 18109.9 | 825.7 | 4450.8 | 420.2 | 5394.3 | 1037.7 | 4144.7 | 998.9 | 11124.7 |
| Clone-53 | 146.6 | 1756.8 | 54.8 | 300.2 | 1.0 | 139.8 | 71.5 | 656.4 | 121.1 | 3792.7 |
| Clone-54 | 1309.3 | 17059.0 | 335.1 | 9825.8 | 372.3 | 139.3 | 471.4 | 7796.5 | 631.3 | 2277.3 |
| Clone-55 | 567.5 | 3050.9 | 76.6 | 1806.3 | 79.9 | 677.4 | 136.0 | 3720.0 | 55.7 | 1022.3 |
| Clone-56 | 482.0 | 4271.5 | 76.6 | 724.3 | 64.7 | 276.0 | 129.0 | 3562.8 | 244.0 | 5032.7 |
| Clone-57 | 613.1 | 379.2 | 100.1 | 364.0 | 112.3 | 1508.4 | 86.2 | 2252.5 | 200.5 | 1793.5 |
| Clone-58 | 446.9 | 4121.2 | 202.4 | 1353.5 | 64.2 | 221.0 | 30.8 | 780.7 | 124.1 | 1067.2 |
| Clone-59 | 4321.5 | 24730.0 | 1739.9 | 11739.1 | 769.0 | 2955.3 | 996.0 | 11213.9 | 2447.8 | 11874.1 |
| Clone-60 | 356.4 | 432.6 | 89.6 | 1714.2 | 112.7 | 111.1 | 56.7 | 628.5 | 51.5 | 185.5 |
| Clone-61 | 1407.1 | 17515.1 | 315.0 | 1467.8 | 100.7 | 394.1 | 726.3 | 7347.3 | 1539.4 | 30451.0 |
| Clone-62 | 547.5 | 6738.7 | 169.0 | 1643.0 | 119.7 | 1034.8 | 339.4 | 5091.2 | 404.2 | 5864.2 |
| Clone-63 | 320.4 | 3242.9 | 100.7 | 709.0 | 28.6 | 234.9 | 143.5 | 2734.5 | 443.4 | 9977.3 |
| Clone-64 | 12765.0 | 26635.1 | 1299.3 | 8254.0 | 470.6 | 1816.7 | 1304.8 | 17388.5 | 5464.6 | 19511.4 |
| Clone-65 | 179.9 | 1707.2 | 62.5 | 603.9 | 14.0 | 114.6 | 22.9 | 1523.7 | 160.6 | 6267.0 |
| Clone-66 | 1762.1 | 6185.5 | 751.1 | 2320.5 | 342.4 | 2813.9 | 844.4 | 3093.0 | 1440.9 | 4955.6 |

TABLE 9
Genes Over Expressed in Kidney Tumors (part two)

| | Control | Cancer | Control | Cancer | Control | Cancer | Control | Cancer | Control | Cancer |
|---|---|---|---|---|---|---|---|---|---|---|
| | Patient 01020069 | | Patient 01020106 | | Patient 01020121 | | Patient 01020241 | | Patient lixingan | |
| Mergen ID | Control | Cancer | Control | Cancer | Control | Cancer | Control | Cancer | Control | Cancer |
| Clone-41 | 528 | 6679 | 260 | 14331 | 237 | 8022 | 702 | 151 | 261 | 1543 |
| Clone-42 | 223 | 5098 | 501 | 14400 | 334 | 2846 | 105 | 279 | 244 | 438 |
| Clone-43 | 187 | 1201 | 399 | 3168 | 187 | 1412 | 24 | 126 | 671 | 202 |
| Clone-44 | 220 | 4010 | 387 | 6217 | 183 | 860 | 1399 | 162 | 768 | 1617 |
| Clone-45 | 418.6 | 5399.1 | 2008.3 | 8243.5 | 478.0 | 954.4 | 7890.2 | 9330.8 | | |
| Clone-46 | 384.2 | 3116.1 | 1163.5 | 5273.1 | 217.8 | 277.1 | 2626.5 | 9171.3 | | |
| Clone-47 | 818.2 | 5428.8 | 1545.8 | 10334.6 | 231.5 | 409.2 | 3001.6 | 9650.0 | | |
| Clone-48 | 423.8 | 1860.9 | 327.3 | 1446.8 | 575.9 | 338.1 | 3932.2 | 2425.6 | | |
| Clone-49 | 2546.3 | 23516.9 | 4128.3 | 63021.8 | 5399.1 | 1353.8 | 8834.9 | 65536.0 | | |
| Clone-50 | 256.0 | 1988.2 | 644.6 | 5106.5 | 71.2 | 84.3 | 7998.6 | 3268.3 | | |
| Clone-51 | 1307.7 | 7167.6 | 3377.6 | 11467.2 | 357.5 | 580.1 | 6405.7 | 13204.1 | | |
| Clone-52 | 1334.6 | 5134.2 | 1289.6 | 17276.9 | 188.4 | 876.6 | 1259.7 | 3441.1 | | |
| Clone-53 | 85.9 | 1265.3 | 184.9 | 1603.4 | 50.0 | 92.0 | 2387.0 | 1826.3 | | |
| Clone-54 | 556.0 | 15473.1 | 1534.5 | 26849.8 | 328.3 | 763.2 | 2072.4 | 21004.6 | | |
| Clone-55 | 55.7 | 1180.3 | 397.0 | 1828.2 | 115.7 | 522.0 | 1256.2 | 1391.0 | | |
| Clone-56 | 77.4 | 2134.7 | 393.6 | 3266.6 | 105.1 | 124.3 | 1312.3 | 2408.6 | | |
| Clone-57 | 242.4 | 398.6 | 504.3 | 373.1 | 130.3 | 470.2 | 532.9 | 5001.8 | | |
| Clone-58 | 169.5 | 2464.4 | 350.2 | 4972.1 | 349.4 | 989.2 | 660.1 | 2679.2 | | |
| Clone-59 | 2688.7 | 11641.6 | 2662.5 | 14825.4 | 4031.7 | 3339.3 | 16184.3 | 22141.0 | | |
| Clone-60 | 1.0 | 597.5 | 472.0 | 1459.5 | 395.2 | 4242.0 | 1196.4 | 913.0 | | |
| Clane-61 | 629.4 | 12810.9 | 1467.2 | 8952.9 | 348.8 | 2730.7 | 8440.9 | 4639.6 | | |
| Clone-62 | 274.5 | 3650.4 | 965.2 | 9434.3 | 116.9 | 329.9 | 1975.1 | 2414.4 | | |
| Clone-63 | 347.3 | 2092.6 | 382.8 | 3251.3 | 57.6 | 185.2 | 2425.3 | 5155.8 | | |
| Clone-64 | 2153.4 | 13099.6 | 6553.0 | 44217.3 | 1098.0 | 2149.9 | 22649.6 | 15243.1 | | |
| Clone-65 | 93.3 | 666.4 | 223.8 | 1290.1 | 32.0 | 73.8 | 1761.3 | 1304.4 | | |
| Clone-66 | 1540.9 | 6624.9 | 2147.9 | 8871.6 | 4372.7 | 896.5 | 3421.4 | 12222.8 | | |

### Example 7

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 μg/ml. The antibodies are either monoclonal or polyclonal. The wells are blocked so that non-specific binding of the polypeptide to the well is reduced.

The coated wells are then incubated for >2 hours at room temperature (RT) with a sample containing the polypeptide. Preferably, serial dilutions of the sample should be used to validate results. The plates are then washed three times with deionized or distilled water to remove unbounded polypeptide.

Next, 50 microliters (μl) of specific antibody-alkaline phosphatase conjugate, at a concentration of 25-400 nanogram. (ng), is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 8

### Method of Treating Increased Levels of the Polypeptide

Antisense technology is used to inhibit production of a polypeptide of the present invention. This technology is one example of a method of decreasing levels of a polypeptide, preferably a secreted or membrane-bound form, due to a variety of etiologies, such as cancer.

For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated.

### Example 9

### Method of Treatment Using Gene Therapy

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g. Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P.T. et al., DNA, 7:219-25 (1988)), flanked by the long terminal repeats of the Moloney murine sarcoma virus, is digested with EcoRI and Hindlel and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRi and HindIII fragment are added together, in the presence of T4 DNA ligase. The resulting mixture is maintained under conditions appropriate for ligation of the two fragments. The ligation mixture is then used to transform bacteria HB 101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm² plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 10

### Western Blots of Cell Lines to Understand their Characteristics as Relevant to Cancers

Western analysis was performed in a manner familiar to those experienced in the art but summarized below.

Rabbit IgG against the protein product of Clbne-49 (coding for Unigene#Hs.323477 and described as “solute carrier family 16 (monocarboxylic acid transporters), member 3; monocarboxylate transporter 3 [Homo sapiens]”) was used as the primary antibody. The secondary antibody, obtained from Pierce (Rockford, Ill. USA), was ImmunoPure® goat anti-rabbit IgG, Fc fragment specific and horseradish peroxidase conjugated.

Thirty cell lines were grown to confluency and collected into a lysis buffer. The lysate was centrifuged, the pellet (containing the membrane bound proteins) was collected and resuspened in “Protein Buffer” (50 mN Tris ph8.0, 150 mM NaCl, 10% Triton X10 and protease inhibitors).

Cell lysates were run through an SDS (Sodium Dodecyl Sulfate) PAGE (PolyAcrylamide Gel for Electrophoresis) gel (0.1% SDS, 10%PA, inTris buffer).

The gel's protein pattern was transferred to nitrocellulose paper to produce a Western blot. The blot was hybridized with the primary antibody, washed and then the secondary antibodies were applied. After washing, the horseradish peroxidase reaction was visualized using Amersham Biosciences' ECL™ Western Blotting Detection Reagents (catalogue #RPN2106), a chemiluminescence technique resulting from the HRP-catalyzed breakdown of luminol. The resulting light was detected by autoradiography.

Table 10 summarizes the results. Note that “+++” is a strong positive signal (indicating large amounts of the protein was present), “++” is a mildly positive signal (indicating some amounts of the protein was present but not as much as those indicated by “+++”), “+” represents a very small signal (at the lower limits of detection in this experiment) and “ND” means that no protein was detected (meaning there might be no protein but, more conservatively suggesting any protein was below our limits to detect it).

TABLE 10
Genes Over Expressed in Certain Cell Lines

| Cell Line # | Cell Line | Tissue of origin | Presence |
|---|---|---|---|
| 1 | 293 | Kidney, | +++ |
| | | adno trans | |
| 2 | A2780 | Ovarian | ND |
| 3 | A549 | Lung | ND |
| 4 | AT20 | Ovary | ND |
| 5 | C-33A | Cervical | ND |
| 6 | DLD1 | Colon | ND |
| 7 | DU145 | Prostate | +++ |
| 8 | EKVX | Lung | +++ |
| 9 | GM47 | Glioblastoma | ND |
| 10 | H28 | Lung-SCLC | ND |
| 11 | HCT-116 | Colon | ND |
| 12 | Hela | Cervx | ND |
| 13 | HEPG2 | Liver | +++ |
| 14 | HS578T | Breast | + |
| 15 | HT-29 | Colon | +++ |
| 16 | HTB-47 | kidney | +(+) |
| 17 | HUH-7 | Liver | ND |
| 18 | K562 | Bone marrow | ND |
| 19 | LnCAp | Prostate | +++ |
| 20 | LOX | Melaoma | ND |
| 21 | MCF-7 | Breast | +++ |
| 22 | MDA-231 | Breast | ND |
| 23 | MDA-MB231 | Breast | + |
| 24 | MDA-MB-435 | Breast | + |
| 25 | OVC1.1 | Ovarian | ND |
| 26 | OVC1.4 | Ovarian | ND |
| 27 | SJSA-1 | Bone | ND |
| 28 | SK-OV-3 | Ovarian | ND |
| 29 | U2-OS | Bone | ND |
| 30 | UC729-6 | Lymphocyte | ND |

### Example 11

### Immunohistochemical “Staining” to Establish Tumor Specificity

Immunohistochemical “staining” was performed in a manner familiar to those exeperienced in the art but summarized below.

Rabbit IgG against the protein product of Clone-49 (coding for Unigene#Hs.323477 and described as “solute carrier family 16 (monocarboxylic acid transporters), member 3; monocarboxylate transporter 3 [Homo sapiens]”) was used as the primary antibody. The secondary antibody and detection system used rabbit “ABC Staining System”, obtained from Santa Cruz Biotechnology, Inc (Sanat Cruz, Calif., USA), which uses a performed avidin-biotinylated horseradish peroxidase complex as the detection agent.

Human tumor and adjacent tissue (obtained during surgery) were. fixed in 4% neutral buffer formalin 12-14 hours (according to the size of the tissue) then dehydrated and embodedded in paraffin. The tissue embedded paraffin block was sliced by microtone into sections micron (4μ) thick, adhered onto standard glass slides and stained with hematoxylin and eosin (H&E) for diagnosis and to determine the best orientation of the block that would give optimal positioning for subsequent slices to produce the material used for the immunohistochemistry (explain below).

Serial 5 micron (5μ) sections cut from the block, adhered onto standard glass slides, then passed through a series of solutions of increasing ethanol concentration (the final concentration being 100% ethanol) to remove the paraffin and dehydrate the specimen(s).

Each slides was covered with a solution containing the primary antibody (in an appropriate buffer) and incubated at 4° C. overnight. The following day the slides were washed three times in PBS (phosphate buffered saline) and a solution containing the secondary antibody (in an appropriate buffer) was applied and allowed to incubate at 37° C. for 40 minutes. After washing, the slides were incubated in DAB chromogen (3,3′ Diaminobenzidine) as recommended by the supplier (Vector Laboratories, Burlingame, Calif., USA) to produce brown precipitate at the location of the antibody complex

FIG. 1 shows images of the immunohistochemical stained tissues showing greatly increased presence of the Clone49 protein in the (cell memebrane) of the tumor compared to the lower expression seen in the adjacent but health tissue.

### Example 12

### Over Expression of Genes in Mammalian Cells

Several clones were over expressed in COS-7 cells using the following method. Individual clones (DNA sequences) were ligated into the expression vector pCMV6-XL5 (from Origene Technologies Inc, Rockville, Md.). Each construct (clone- pCMV6-XL5) was mixed with FuGENE 6 (from Roche Catalogue#181443) as directed by the manufacturer (Roche) and incubated at room temperature for 30 minutes. Each construct-FuGENE 6 mixture was then use to transform COS-7 cells (in culture plates containing 5 mls of RPMI medium with no serum. These were then placed in a 5% CO₂ incubator at 37° C. for 8 hours after which the medium was changed to DMEM with 10% fetal calf serum and incubated for another 48 hours.

### Example 13

### Production of Monoclonal Antibodies to Gene Product

Monoclonal antibodies were made to the gene products of several of the clones using cells carrying the transgene (described in Example 12) using the method of subtractive immunization described below. Practitioners are particularly directed to Rao A. G. and Sleister H. M “Subtractive immunization: a tool for the generation of discriminatory antibodies to proteins of similar sequence” JOURNAL OF IMMUNOLOGICAL METHODS, 261 (2002) 213-220. It is to be understood that this invention is not limited to the particular methodology, protocols, and reagents described, as these may vary.

Briefly, cyclophosphamide-induced immunosuppression of several 5-6 week old BALB/c mice inoculated with the non-transgenic COS-7 cells was followed by immunization with COS-7 cells expressing the transprotein (carrying the gene to one of the clones). Tolerization was achieved by subcutaneous (s.q.) injections consisting of COS-7 cells (2×10⁶ cells in 0.3 millileters of sterile PBS) followed by peritoneum (i.p.) injections of cyclophosphamide (Sigma catalog#C7397 100 mg per kg mouse in a volume of 100 microliters) 24 and 48 hours later. This tolerization cycle was repeated two weeks later. After a further two weeks (at the end of the tolerization stage after the final cyclophosphamide treatment) tail blood was collected for subsequent analysis. One week later the mice were injected (i.p.) with COS-7 cells carrying the transgene (2×10⁶ cells in 0.3 millileters of sterile PBS) produced by the methods described above in Example 12. This immunization cycle was repeated three weeks later. After a further three weeks (at the end of the immunization stage—after the final transgenic COS-7 treatment) tail blood was collected for subsequent analysis. The pairs of tail blood sera (post tolerization and post immunization) were evaluated by ELISA to determine reactivity to non-transgenic COS-7 and transgenic COS-7 cells. This data were used to choose the mouse with the greatest differential reactivity (to the transgenic COS-7 cells veruses the non-transgenic COS-7 cells) for production of monoclonal antibodies (Mabs) using techniques that are well known to those experienced in the art.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the sequence listing submitted herewith and the corresponding computer readable form are both incorporated herein by reference in their entireties.

All publications, patents and patent applications mentioned in this specification are herein expressly incorporated by reference in their entirety to the same extent as if each individual publication, patent or patent application was specifically and individually indicated to be incorporated by reference in its entirety.

The foregoing description of preferred embodiments of the invention has been presented by way of illustration and example for purposes of clarity and understanding. It is not intended to be exhaustive or to limit the invention to the precise forms disclosed. It will be readily apparent to those skilled in the art in light of the teachings of this invention that many changes and modification may be made thereto without departing from the spirit of the invention. It is intended that the scope of the invention be defined by the appended claims and their equivalents.

We claim:

1. A method of breast cancer diagnosis comprising detecting over expression of a gene of a protein found in breast tissue selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDR/TAP) and Type I transmembrane receptor (seizure-related protein).

2. A method as in claim 1, wherein the gene comprises a sequence selected from the group consisting of SEQ ID Nos 1-154 in the file “14dec03.ST25.txt” (generated by PatentIn3.2) attached to this application and incorporated by reference.

3. A method as in claim 1, wherein detecting over expression comprises a method selected from the group consisting of detecting a level of mRNA expression, detecting a level of protein expression, and probing for a polypeptide encoded by the gene.

4. A method as in claim 3, wherein the over expressed protein comprises an amino acid sequence encoded by a gene selected from the group of genes having nucleic acid sequences of SEQ ID Nos 1-154 in the file “14dec03.ST25.txt” (generated by PatentIn3.2) attached to this application and incorporated by reference.

5. A method of breast cancer treatment comprising blocking over expression of a gene encoding a secreted or membrane-bound protein found in breast tissue selected from the group consisting of SEQ ID Nos 1-154 in the file “14dec03.ST25.txt” (generated by PatentIn3.2) attached to this application and incorporated by reference.

6. A method of treatment as in claim 5, wherein the gene comprises a sequence selected from the group consisting of SEQ ID Nos 1-154 in the file “14dec03.ST25.txt” (generated by PatentIn3.2) attached to this application and incorporated by reference.

7. A method as in claim 5, wherein the method of treatment comprises a method selected from the group consisting of blocking expression of an mRNA encoded by the gene, blocking translation of the mRNA encoded by the gene, blocking expression of a protein encoded by the gene, blocking the function of the expressed protein, promoting the degradation of the expressed protein and using the protein as a tag to deliver treatment.

8. A method as in claim 7, wherein the method of treatment comprises blocking an mRNA, polypeptide or protein comprising a nucleotide or amino acid sequence encoded by a gene selected from the group of genes having nucleic acid sequences of SEQ ID Nos 1-154 in the file “14dec03.ST25.txt” (generated by PatentIn3.2) attached to this application and incorporated by reference.

9. A method as in claim 1, wherein the over expressed protein is secreted.

10. A method as in claim 1, wherein the over expressed protein is membrane-bound.

11. A method of lung cancer diagnosis comprising detecting over expression of a gene of a protein found in lung tissue selected from the group consisting of over expressed in lung of hypothetical protein, amino acid transporter 2, carboxypeptidase M, and putative G-protein coupled receptor.

12. A method as in claim 11, wherein the gene comprises a sequence selected from the group consisting of SEQ ID Nos 155-184 in the file “14dec03.ST25.txt” (generated by PatentIn3.2) attached to this application and incorporated by reference.

13. A method as in claim 11, wherein detecting over expression comprises a method selected from the group consisting of detecting a level of mRNA expression, detecting a level of protein expression, and probing for a polypeptide encoded by the gene.

14. A method as in claim 13, wherein the over expressed protein comprises an amino acid sequence encoded by a gene selected from the group of genes having nucleic acid sequences of SEQ ID Nos 155-184 in the file “14dec03.ST25.txt” (generated by PatentIn3.2) attached to this application and incorporated by reference.

15. A method of lung cancer treatment comprising blocking over expression of a gene encoding a secreted or membrane-bound protein found in lung tissue selected from the group consisting of SEQ ID Nos 155-184 in the file “14dec03.ST25.txt” (generated by PatentIn3.2) attached to this application and incorporated by reference.

16. A method of treatment as in claim 15, wherein the gene comprises a sequence selected from the group consisting of SEQ ID Nos 155-184 in the file “14dec03.ST25.txt” (generated by PatentIn3.2) attached to this application and incorporated by reference.

17. A method as in claim 15, wherein the method of treatment comprises a method selected from the group consisting of blocking expression of an mRNA encoded by the gene, blocking translation of the mRNA encoded by the gene, and blocking expression of a protein encoded by the gene, blocking the function of the expressed protein, promoting the degradation of the expressed protein and using the protein as a tag to deliver treatment.

18. A method as in claim 17, wherein the method of treatment comprises blocking an mRNA, polypeptide or protein comprising a nucleotide or amino acid sequence encoded by a gene selected from the group of genes having nucleic acid sequences of SEQ ID Nos 155-184 in the file “14dec03.ST25.txt” (generated by PatentIn3.2) attached to this application and incorporated by reference.

19. A method as in claim 11, wherein the over expressed protein is secreted.

20. A method as in claim 11, wherein the over expressed protein is membrane-bound.

21. A method of colon cancer diagnosis comprising detecting over expression of a gene of a protein found in colon tissue selected from the group consisting of hypothetical protein (colon), cadherin, formyl peptide receptor 1, AE-binding protein 1, solute carrier family 21 (organic anion transporter) member 12, secreted phosphoprotein 1 (osteopontin, bone sialoprotein I), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

22. A method as in claim 21, wherein the gene comprises a sequence selected from the group consisting of SEQ ID Nos 185-246 in the file “14dec03.ST25.txt” (generated by PatentIn3.2) attached to this application and incorporated by reference.

23. A method as in claim 21, wherein detecting over expression comprises a method selected from the group consisting of detecting a level of mRNA expression, detecting a level of protein expression, and probing for a polypeptide encoded by the gene.

24. A method as in claim 23, wherein the over expressed protein comprises an amino acid sequence encoded by a gene selected from the group of genes having nucleic acid sequences of SEQ ID Nos 185-246 in the file “14dec03.ST25.txt” (generated by PatentIn3.2) attached to this application and incorporated by reference.

25. A method of colon cancer treatment comprising blocking over expression of a gene encoding a secreted or membrane-bound protein found in colon tissue selected from the group consisting of SEQ ID Nos 185-246 in the file “14dec03.ST25.txt” (generated by PatentIn3.2) attached to this application and incorporated by reference.

26. A method of treatment as in claim 25, wherein the gene comprises a sequence selected from the group consisting of SEQ ID Nos 185-246 in the file “14dec03.ST25.txt” (generated by PatentIn3.2) attached to this application and incorporated by reference.

27. A method as in claim 25, wherein the method of treatment comprises a method selected from the group consisting of blocking expression of an mRNA encoded by the gene, blocking translation of the mRNA encoded by the gene, and blocking expression of a protein encoded by the gene, blocking the function of the expressed protein, promoting the degradation of the expressed protein and using the protein as a tag to deliver treatment.

28. A method as in claim 27, wherein the method of treatment comprises blocking an mRNA, polypeptide or protein comprising a nucleotide or amino acid sequence encoded by a gene selected from the group of genes having nucleic acid sequences of SEQ ID Nos 185-246 in the file “14dec03.ST25.txt” (generated by PatentIn3.2) attached to this application and incorporated by reference.

29. A method as in claim 21, wherein the over expressed protein is secreted.

30. A method as in claim 21, wherein the over expressed protein is membrane-bound.

31. A method of kidney cancer diagnosis comprising detecting over expression of a gene of a protein found in kidney tissue selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150), alpha polypeptide), sema domain seven thrombospondin repeats (type 1 and type 1-like) transmembrane domain (TM) and short cytoplasmic domain (semaphorin) 5B, guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

32. A method as in claim 31, wherein the gene comprises a sequence selected from the group consisting of SEQ ID Nos 247-385 in the file “14dec03.ST25.txt” (generated by PatentIn3.2) attached to this application and incorporated by reference.

33. A method as in claim 31, wherein detecting over expression comprises a method selected from the group consisting of detecting a level of mRNA expression, detecting a level of protein expression, and probing for a polypeptide encoded by the gene.

34. A method as in claim 33, wherein the over expressed protein comprises an amino acid sequence encoded by a gene selected from the group of genes having nucleic acid sequences of SEQ ID Nos 247-385 in the file “14dec03.ST25.txt” (generated by PatentIn3.2) attached to this application and incorporated by reference.

35. A method of kidney cancer treatment comprising blocking over expression of a gene encoding a secreted or membrane-bound protein found in kidney tissue selected from the group consisting of SEQ ID Nos 247-385 in the file “14dec03.ST25.txt” (generated by PatentIn3.2) attached to this application and incorporated by reference.

36. A method of treatment as in claim 35, wherein the gene comprises a sequence selected from the group consisting of SEQ ID Nos 247-385 in the file “14dec03.ST25.txt” (generated by PatentIn3.2) attached to this application and incorporated by reference.

37. A method as in claim 35, wherein the method of treatment comprises a method selected from the group consisting of blocking expression of an mRNA encoded by the gene, blocking translation of the mRNA encoded by the gene, blocking expression of a protein encoded by the gene, blocking the function of the expressed protein, promoting the degradation of the expressed protein and using the protein as a tag to deliver treatment.

38. A method as in claim 37, wherein the method of treatment comprises blocking an mRNA, polypeptide or protein comprising a nucleotide or amino acid sequence encoded by a gene selected from the group of genes having nucleic acid sequences of SEQ ID Nos 247-385 in the file “14dec03.ST25.txt” (generated by PatentIn3.2) attached to this application and incorporated by reference.

39. A method as in claim 31, wherein the over expressed protein is secreted.

40. A method as in claim 31, wherein the over expressed protein is membrane-bound.

41. An antibody or binding portion of an antibody that specifically binds a protein found in breast tissue, wherein the protein is selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDR/TAP) and Type I transmembrane receptor (seizure-related protein).

42. A method of breast cancer diagnosis or prognosis comprising contacting an antibody or binding portion of an antibody of claim 41, with the over expressed protein for which the antibody or binding portion thereof is specific.

43. A method of breast cancer treatment comprising blocking an over expressed protein selected from the group consisting of ATP transporter protein, GPCR (G-protein coupled receptor), GPCR (G-protein coupled receptor) 14, hypothetical protein (breast), unc-93, bone marrow stromal cell antigen 2, cadherin EGF LAG seven-pass G-type receptor 1 flamingo (Drosophila) homolog, carcinoembryonic antigen-related cell adhesion molecule 3, carcinoembryonic antigen-related cell adhesion molecule 6, carcinoembryonic antigen-related cell adhesion molecule 8, claudin 7, cleft lip and palate associated membrane transmembrane protein 1, fibroblast growth factor receptor 3, heme oxygenase (decycling) 1, immediate early response 3, mucin 1 transmembrane, NG22, phosphatidylinositol glycan (class Q), similar to CGI-78 protein, clone MGC:4880 IMAGE:3457593, solute carrier family 1 (member 4), solute carrier family 6 (member 8), tetraspan 1, transporter 1 ATP-binding cassette sub-family B (MDR/TAP) and Type I transmembrane receptor (seizure-related protein), with an antibody or binding portion thereof specific for said protein.

44. An antibody or binding portion of an antibody that specifically binds a protein found in lung tissue, wherein the protein is selected from the group consisting of over expressed in lung of hypothetical protein, amino acid transporter 2, carboxypeptidase M, and putative G-protein coupled receptor.

45. A method of lung cancer diagnosis or prognosis comprising contacting an antibody or binding portion of an antibody of claim 44, with the over expressed protein for which the antibody or binding portion thereof is specific.

46. A method of lung cancer treatment comprising blocking an over expressed protein selected from the group consisting of over expressed in lung of hypothetical protein, amino acid transporter 2, carboxypeptidase M, and putative G-protein coupled receptor, with an antibody or binding portion thereof specific for said protein.

47. An antibody or binding portion of an antibody that specifically binds to a protein found in colon tissue, wherein the protein is selected from the group consisting of hypothetical protein (colon), cadherin, formyl peptide receptor 1, AE-binding protein 1, solute carrier family 21 (organic anion transporter) member 12, secreted phosphoprotein 1 (osteopontin, bone sialoprotein I), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

48. A method of colon cancer diagnosis or prognosis comprising contacting an antibody or binding portion of an antibody of claim 47, with the over expressed protein for which the antibody or binding portion thereof is specific.

49. A method of colon cancer treatment comprising blocking an over expressed protein selected from the group consisting of hypothetical protein (colon), cadherin, formyl peptide receptor 1, AE-binding protein 1, solute carrier family 21 (organic anion transporter) member 12, secreted phosphoprotein 1 (osteopontin, bone sialoprotein I), Type I transmembrane protein Fn14, hypoxia-inducible protein 2, alpha-2-glycoprotein 1 zinc, chitinase 3-like1 (cartilage glycoprotein-39) and triggering receptor expressed on myeloid cells 2.

50. An antibody or binding portion of an antibody that specifically binds to a protein found in kidney tissue, wherein the protein is selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150), alpha polypeptide), sema domain seven thrombospondin repeats (type 1 and type 1-like) transmembrane domain (TM) and short cytoplasmic domain (semaphorin) 5B, guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.

51. A method of kidney cancer diagnosis or prognosis comprising contacting an antibody or binding portion of an antibody of claim 50, with the over expressed protein for which the antibody or binding portion thereof is specific.

52. A method of kidney cancer treatment comprising blocking an over expressed protein selected from the group consisting of scavenger receptor class B, adenosine receptor A3, CD97 antigen, APO E-C2 gene, basement membrane-induced gene, integrin alpha 5 (fibronectin receptor, alpha polypeptide), peptide transporter 3, hypothetical protein FLJ22341, solute carrier family 16 (monocarboxylic acid transporters), interleukin 10 receptor alpha, FXYD domain-containing ion transport regulator 5, retinoic acid receptor responder (tazarotene induced) 2, integrin alpha X (antigen CD11C (p150), alpha polypeptide), sema domain seven thrombospondin repeats (type 1 and type 1-like) transmembrane domain (TM) and short cytoplasmic domain (semaphorin) 5B, guanylate binding protein 1 interferon-inducible 67 kD, leukocyte immunoglobulin-like receptor subfamily B, discoidin domain receptor family member 2, caveolin 1 caveolae protein 22 kD, chloride intracellular channel 1, CD36 antigen (collagen type I receptor, thrombospondin recep), small inducible cytokine A4 (homologous to mouse Mip-1b), lysosomal-associated multispanning membrane protein-5, integrin beta 2 antigen CD18 (p95) lymphocyte funtion-assoc, chemokine (C-X-C motif) receptor 4 (fusin), CD2 antigen (p50) sheep red blood cell receptor and epithelial membrane protein 3.
